(19) United States
(12) Patent Application Publication
(10) Pub. No.: US 2016/0067217 A1
(43) Pub. Date: Mar. 10, 2016

(54) METABOLIC SYNDROME TREATMENT

(71) Applicant: ARKAY Therapeutics, LLC, East Windsor, NJ (US)

(72) Inventor: Ravi Seshagirirao KUMAR, East Windsor, NJ (US)

(21) Appl. No.: 14/848,972

(22) Filed: Sep. 9, 2015

Related U.S. Application Data
(60) Provisional application No. 62/047,766, filed on Sep. 9, 2014.

(51) Int. Cl. A61K 31/415; A61K 31/155; A61K 38/28; A61K 31/40; A61K 31/522; A61K 38/26; A61K 31/41

(52) U.S. Cl. CPC A61K 31/415; A61K 38/26; A61K 31/155; A61K 31/41; A61K 31/40; A61K 31/522; A61K 38/28

(57) ABSTRACT

Formulations and methods of providing an orally-active anti-metabolic disease Fixed Dose Combinations (FDC) for use as personalized medicine to treat different components of the Metabolic Syndrome or Insulin resistance syndrome such as Type II diabetes, Hypertension, Hyperlipidemia and Obesity are disclosed. Pharmaceutical compositions of anti-inflammatory centric drug formulations and methods comprising of NSAIDS in general and selective Cox-2 inhibitors in particular and one or more anti-T2DM or anti-hypertensive or anti-hyperlipidemic or anti-obesity drugs formulated to exhibit pre-determined modified release kinetics to achieve therapeutic as well as kinetic synergies are disclosed.

### FIELD OF INVENTION

The invention relates to development of drugs to treat metabolic syndrome targeting components of the complex pathophysiology.

### BACKGROUND OF THE INVENTION

Metabolic diseases in general and Type II diabetes [(T2DM or T2D or Type 2 Diabetes or Non-insulin dependent mellitus (NIDDM)] in particular are a complex, multigene and multifactorial disease. Metabolic diseases such as hyperlipidemia, obesity and hypertension as well as environmental factors contribute to this disease. Cardiometabolic risk factor clusters (CMRFC) such as diabetes, hyperlipidemia, hypertension and overweight/obesity often cluster together in the same individual (Garber, A. J. et al. 2013). The prevalence of these risk factors is increasing significantly for all sociodemographic groups and it is putting an enormous economic burden on the society. Currently over one-fourth (over 70 million) of the U.S. population live with cardiovascular (CV) disease along with cardiometabolic risk factors. The economic impact of CV disease is enormous. In 2005, it was estimated to be over $242B in direct medical expenses and over $152B in indirect medical costs including lost productivity, resulting in a total estimated cost of $395B in the U.S. alone (Sullivan, P. W. et al. 2007). CV disease is the leading cause of death resulting in an estimated 40% (>840,000) of all the deaths. Over 225 million people worldwide and over 26 million people in the USA alone suffer from Type II diabetes. In 2013, the annual cost of clinical management of this disease was over $245B ($176B for direct medical costs and $69B in reduced productivity) (Statistics about diabetes, ADA, 2014). Type II diabetes was the seventh leading cause of death in the U.S. in 2010 (Mortality data, 2010, CDC). According to GB Research (Global Business Intelligence), the global Type II diabetes market will grow from $20.4B in 2012 to $38.8B in 2019. The U.S. market will more than double, from $12.7B in 2012 to $27.2B in 2019 (GBI Research, 2013). The world market for metabolic syndrome is estimated to be $72.4B by 2018 (biovision.com).

Insulin resistance is a pathological hallmark of metabolic syndrome in general and Type II diabetes in particular. Type II diabetes continues to be an unmet medical need due to a number of factors including: the idiopathic nature of the disease, complex pathophysiology attributed to auto-immune and pro-inflammatory components, and comorbidities such as hyperlipidemia, obesity and high blood pressure (Erik, P. et al. 2009). Complexity of the disease contributes to manifestation of T2DM in multiple and diverse pathophysiological conditions such a way that the disease constitutes a unique pathophysiological phenomenon in each patient. Complexity of the disease in combination with unique metabolic profile and life style of each patient or group of patients contribute to lack of adequate efficacy with currently marketed Type II diabetes drugs (American College of Physicians, 2012). Moreover, Type II diabetes patients who are on current treatment regimens, continue to be vulnerable for complications such as diabetic retinopathy, skin ulcers, risk of coronary heart disease (CHD), stroke, chronic kidney disease (CKD), diabetic peripheral neuropathy, diabetic vasculopathy etc.

Activation of inducible Cyclooxygenase, Cox-2 or activation of constitutive Cox-2 plays a critically important role in the initiation of obesity-triggered inflammation. A link between elevation of blood glucose levels and activation of Cox-2 in pancreatic beta cells is well established. High glucose-induced PGE2 causes reduction in the beta cell mass by inhibiting its proliferation as well as inducing apoptosis of beta cells (Oshima, H. et al 2006). Indomethacin, a non-selective Cyclooxygenase inhibitor prevented HFD-(high fat diet)-induced obesity and insulin resistance in C57BL/6J mice (Fjaere E. et al. 2014). Treatment with NSAIDs (Non-steroidal anti-inflammatory drugs) such as Celecoxib and Salsalate have been shown to restore systemic insulin sensitivity in both translational preclinical models as well as in obese patients with T2DM (Goldfine, A. B. et al. 2013; Gonzalez-Ortiz, et al. 2005). High blood glucose activates Cox-2 in pancreatic beta cells and contributes to beta cell dysfunction. Treatment with NS-398 (a selective Cox-2 inhibitor) reverses beta cell dysfunction (Tian, V. F. et al. 2014) presumably by reducing PGE2-mediated beta cell apoptosis. Obesity-triggered inflammation due high blood sugar levels results in non-alcoholic hepatic steatosis which is a pathological hallmark of insulin resistance. Non-alcoholic steatohepatitis (NASH) is a condition that coexists with T2DM. As shown in FIG. 7, Celecoxib reverses steatohepatitis as well as inflammation in HFD-induced Wistar rat NASH model (Chen, J. et al. 2011). Over expression of NAG-1/GDF-15 (NSAIDs-activated gene-1) has been shown to improve glycemic parameters and prevent development of obesity by increasing thermogenesis, lipolysis and oxidative metabolism in obese C57BL/6J mice (Chrysovergis, K. et al. 2014). Activation of inducible form of Cox-2 plays a critically important role in the initiation of cellular dysfunctions including: adipocyte dysfunction, pancreatic beta islet cell dysfunction and macrophage dysfunction. Cellular dysfunctions contribute to development of insulin resistance and systemic glucose intolerance. As shown in FIG. 4, Cox-2 deletion in C57BL/6J obese mice reduces blood glucose levels (Fujta et al. 2007). More importantly, in the same translational preclinical model, selective Cox-2 inhibitor, Celecoxib reduces HbA1c levels, improves glucose tolerance and elevates insulin levels (Fujita, H. et al. 2007). Selective Cox-2 inhibitors such as Celecoxib and Mesulid restore insulin sensitivity, reduce oxidative stress and reverse low-grade inflammation in male Sprague Dawley rats fed with High fructose diet or High fat diet (HFD) (Hsieh, P. et al. 2009; Liu, T. et al. 2009). They both reduced time-dependent increases in plasma insulin, 8-isoprostanes, leptin levels, and reversed increase in hepatic triglycerides. Celecoxib also restored insulin sensitivity in a small study of 12 obese patients (Gonzalez-Ortiz, et al. 2005). Based on the preclinical and the clinical data, treating the underlying inflammatory component of the complex pathophysiology of type 2 diabetes with an anti-inflammatory-centric therapy represents a shift in the paradigm of clinically managing T2DM efficiently.

Renin-Angiotensin system (RAS) exists in pancreatic beta cells and Angiotensin II is pro-inflammatory in pancreas and activates pro-inflammatory cytokine IL-1 beta. Ang II-mediated Islet cell inflammation triggers beta cell dysfunction contributing to Pancreatic beta cell exhaustion and decompensation (Sauter, N. et al. 2015). This occurs independent of vasoconstriction because sub-hyperstensve dose of Valsartan (1 mg/Kg/day) improves impaired glucose tolerance with no effect on the systolic blood pressure in C57BL/6J obese mice. Blockade of Ang II with an ARB (Angiotensin receptor blocker) such as Valsartan improves glucose tolerance (FIG. 9, Cole, B. K. et al. 2010) as well as restores not only beta cell dysfunction but also enhances blood flow as a result of vasodilation. As shown in FIG. 8, Metformin, which is considered the gold standard for the treatment of T2DM ameliorates not only HFD-induced insulin resistance but also improves glucose tolerance in C57BL/6J diet-induced obesity (DIO) model (Matsui, Y. et al. 2010; Woo, S. et al. 2014).

A number of publications have supported the importance of adipocytes and inflammation for the development of insulin resistance. For example, JNK-1 deficiency in adipocytes suppressed HFD-(High fat diet)-induced insulin resistance in the liver due to suppression of JNK-dependent suppression of IL-6 (Sabio, C. et al. 2008), adipocyte-specific deletion of Glut4 or over expression of MCP-1 results in systemic insulin resistance (Qi, L. et al. 2009), TNF-alpha deficiency improved insulin sensitivity in diet-induced obesity and in Lepob/ob model of obesity, neutralization of TNF-alpha in obese fa/fa rats ameliorated insulin resistance (Hotamisiligil, G. S. et al. 1995). Elevated IL-1 beta, IL-6 and CRP are predictive of development of T2DM (Visser, M. et al. 1999) and TLR4 knock-out mice were protected from inflammation and insulin resistance (Shi, H. et al. 2006). Therefore, the lack of adequate efficacy and lack of adequate overall clinical benefit from currently marketed hypoglycemic drugs is due to their inability to suppress the pro-inflammatory components of the complex pathophysiology of initiation and maintenance of systemic insulin resistance. TZDs and statins do have an inherent yet very modest anti-inflammatory capacity which contributes to their therapeutic efficacy. Therefore, suppression of obesity-triggered chronic low-grade inflammation with an anti-inflammatory drug such as Cox-2 selective inhibitor is anticipated to treat the impaired glucose homeostasis in T2DM patients by enhancing the efficacy of hypoglycemic drugs with an additive or synergistic effect. Inhibition of inflammation is also anticipated to reduce the severity of diabetes-related complications.

Inflammation is a critical component of the pathophysiology of not only Type II diabetes but also the clinically relevant comorbidities as illustrated in FIG. 2. More importantly, pro-inflammatory signals contribute to initiation and maintenance of complications associated with Type II diabetes such as diabetic retinopathy, skin ulcers, risk of coronary heart disease (CHD), stroke, chronic kidney disease (CKD), diabetic peripheral neuropathy, diabetic vasculopathy etc. Pro-inflammatory signals determine the severity and duration of diabetes-related complications. An anti-inflammatory drug, Salsalate has been shown to reduce glycosylated hemoglobin A1c by an average of 0.37% in a clinical study conducted by Harvard Medical School (Goldfine, A. et al. 2013). There's a direct correlation between elevation in the pro-inflammatory biomarkers and impaired glucose metabolism. Statins and Thiazolidinediones (TZDs) reduce the risk of developing diabetes by consistently lowering the inflammatory markers (Deans K. A. and Sattar, N. (2006). Therefore, for efficient clinical management, it is important to treat cardiometabolic diseases including Type II diabetes not as an individual disease but as a metabolic syndrome with a complex pathophysiology with a strong pro-inflammatory component. For example, to achieve sufficient therapeutic efficacy in Type II diabetes patients who present wide-ranging challenges and metabolic profiles, it is indeed necessary to treat with a pro-inflammatory-centric combination of drugs that correct not only the impaired glucose homeostasis but also attenuate clinically relevant comorbidities and more importantly reduce the severity of diabetes-related complications. Instead of treating patients with metabolic syndrome as one homogeneous population with just one drug, it is necessary to treat patients with an anti-inflammatory drug in combination with anti-metabolic disease drugs with known efficacy and safety profile.

Patients stratified into groups for lack of adequate clinically favorable response to currently marketed drugs to treat metabolic diseases will be selected to test different Fixed Dose Combinations (FDC) formulations. Combination of drugs custom-formulated to provide therapeutic benefit to a specific stratified group of patients is anticipated to have therapeutic as well as kinetic synergy. More importantly these drugs would block the pro-inflammatory components of some of the comorbidities as well as attenuate the severity of complications such as Chronic kidney disease, diabetic neuropathy, diabetic nephelopathy, skin ulcers etc. Inclusion of an anti-inflammatory drug in every potential FDC formulation would result in enhancing therapeutic efficacy of Type II diabetes drugs by reducing therapeutic dose range such as minimal effective dose (MED), maximal tolerated dose (MTD) and the maintenance dose. Therefore, blockade of the pro-inflammatory component would contribute to sparing or reducing the severity or duration of some of the adverse side effects. Pro-inflammatory-centric anti-metabolic syndrome FDC formulations would result in the development of drugs that differentiate clinically as well as mechanistically from the currently available drugs.

The prevalence of prediabetes is 3 times more than that of Type II diabetes. American Diabetes Association reported that in 2012, there were 86 million Americans 20 or older had prediabetes (Statistics about diabetes, ADA, 2014). Insulin resistance as a result of failing pancreatic compensation due to excessive body weight or obesity, impaired glucose tolerance, impaired fasting glucose and symptomatic metabolic syndrome are the pathological hallmarks of prediabetes (American College of Physicians, 2012). Currently, there are no drugs that prevent or slow down progression of prediabetes into insulin resistance or metabolic syndrome or incidence of new-onset T2DM.

Renin-Angiotensin System (RAS) exists in the pancreatic islets (Leung, P. S. 2012; Andraws, R. and D. L. Brown, 2007). Blockade of the RAS particularly with Angiotensin II receptor blockers (ARBs) and ACE (Angiotensin-converting enzyme) inhibitors has been shown to prevent the incidence or onset of Type II diabetes in patients with impaired glucose tolerance (IGT) (Van der Zijl, N. R. et al. 2011). Moreover, in normotensive patients with impaired glucose tolerance, ARBs increase glucose-dependent insulin secretion and enhance insulin sensitivity (McMurray, et al. 2010). Anti-inflammatory FDC formulations that combine anti-type II diabetes drugs with ARBs and ACE inhibitors will have tremendous therapeutic potential in terms of reducing the number of prediabetes patients who are on a certain path to positive clinical diagnosis for Type II diabetes.

It is hypothesized that in normotensive prediabetes and Type II diabetes, Anti-inflammatory FDC formulations that includes sub therapeutic doses of ARBs and ACE inhibitors would enhance therapeutic efficacy of Type II diabetes drugs by reducing therapeutic dose range [minimal effective dose (MED), maximal tolerated dose (MTD) and the maintenance dose]. Therefore, blockade of the pro-inflammatory and RAS components would contribute to sparing or reducing the severity or duration of some of the adverse side effects associated with Type II diabetes drugs.

There is a continuing need for compounds, compositions, formulations and methods to treat metabolic syndrome.

This Invention has the unique feature of personalized medicine with a custom formulation strategy to combine anti-metabolic disease drugs with anti-inflammatory drugs to reduce the pill burden and to achieve therapeutic as well as kinetic synergies resulting in better efficacy, safety and compliance.

Formulation strategy described in this invention is aimed at providing patients and physicians better choice of drugs.

### OBJECTS AND SUMMARY OF THE INVENTION

It is an object of the invention to provide methods, compositions and formulations to treat Metabolic Syndrome or Insulin resistance syndrome.

It is a further object of the invention to provide broad-spectrum personalized drugs to treat metabolic syndrome including metabolic diseases such as Type II diabetes, hypertension, hyperlipidemia and obesity.

It is a further object of the invention to provide methods of treatment of not just the primary disease but also the comorbidities, risk factors and diabetes-related complications.

It is a further object of the invention to provide methods to treat prediabetes by preventing or delaying the new incidence or onset of Type II diabetes.

In accordance with the above objects and others, the present invention relates to the methods for treating patients with metabolic syndrome with personalized medicines.

The invention is directed in part to a method of providing an orally-active anti-metabolic disease Fixed Dose Combinations (FDC) for use as personalized medicine to treat different components of the Metabolic Syndrome or Insulin resistance syndrome such as Type II diabetes, Hypertension, Hyperlipidemia and Obesity.

The invention is further directed in part to the inclusion of an orally-active anti-inflammatory drug as part of the composition of the drug FDC.

The invention is further directed in part to the inclusion of an orally-active anti-inflammatory drug, at least one anti-diabetic drug and blockers of Renin-Angiotensin System (RAS) as part of the composition of the FDC to prevent the incidence or delay the onset of Type II diabetes.

The invention is also directed in part to formulations and methods in which one or more orally-active anti-inflammatory drugs will be combined with the orally-active anti-metabolic disease drugs in FDC ratios to achieve therapeutic as well as the kinetic synergy.

In certain embodiments, the invention is further directed in part to the use of more than one and as many as five (or more) orally-active anti-metabolic and anti-inflammatory FDC.

Certain embodiments of the invention are directed to the delivery of FDC of orally-active anti-inflammatory drugs and anti-metabolic disease drugs as a multi-layer matrix of immediate release (IR) and extended release (ER) formulations. In certain embodiments of the invention, the delivery of FDC of orally-active anti-inflammatory drugs and anti-metabolic disease drugs is accomplished via a delayed release (DR) formulation. In certain preferred embodiments of the invention, the pharmaceutical formulations of the invention utilize a combination of immediate release, extended release and/or delayed release for the orally-active anti-inflammatory drugs and anti-metabolic disease drugs included in the FDC formulation.

Further embodiments of the invention are directed in part to the delivery of FDC of orally-active anti-inflammatory drugs and anti-metabolic disease drugs as a multi-layer matrix of IR and DR formulations along with one or more drug administered parentarally (intravenous, subcutaneous, intramuscular or intramedullary injection).

Further embodiments of the invention are directed in part to the delivery of FDC of orally-active anti-inflammatory drugs and anti-metabolic disease drugs as a multi-layer matrix of IR and DR formulations along with one or more drug administered transdermally or nasally.

In certain embodiments, the invention is directed in part to the treatment with an anti-inflammatory-centric FDC to treat Type II diabetes component of metabolic syndrome or insulin-resistant syndrome to patients in need of said therapy.

The invention is further directed in part to treatment with an anti-inflammatory-centric FDC to treat hypertension or high blood pressure component of metabolic syndrome or insulin-resistant syndrome.

The invention is further directed in part to treatment with an anti-inflammatory-centric FDC to treat hyperlipidemia component of metabolic syndrome or insulin-resistant syndrome.

The invention is further directed to treatment with an anti-inflammatory-centric FDC to treat excessive bodyweight or obesity component of metabolic syndrome or insulin-resistant syndrome.

The invention is further directed in part to a pharmaceutical formulation of FDC of drugs to treat metabolic syndrome or insulin-resistant syndrome in the form of multi-layered monolithic tablets.

The invention is further directed in part to a pharmaceutical formulation of FDC of drugs to treat metabolic syndrome insulin-resistant syndrome in the form of core tablet-in-tablet or multi-disc tablets.

The invention is further directed in part to a pharmaceutical formulation of FDC of drugs to treat metabolic syndrome insulin-resistant syndrome in the form of beads inside a capsule.

The invention is further directed in part to the use of more than one and as many as five (or more) orally-active drug FDC of IR, IR and DR, and IR and ER formulations for preventing or slowing down prediabetes in the form of multi-layered monolithic tablet or core tablet-in-tablet/multi-disc tablets or beads inside a capsule or tablets inside a capsule.

In certain preferred embodiments, the present invention is directed in part to pharmaceutical formulation for the treatment of metabolic syndrome comprising a therapeutically effective amount(s) of an active agent selected from the group consisting of at least one anti-diabetic drug; or a therapeutically effective amount of at least one anti-hypertensive drug; or a therapeutically effective amount of at least one anti-obesity drug; or a therapeutically effective amount of at least one drug for the treatment of hyperlipidemia; or a combination of any of the foregoing; and a therapeutically effective amount of at least one orally active anti-inflammatory drug. In certain preferred embodiments, the pharmaceutical formulation comprises a therapeutically effective amount(s) of a drug selected from the group consisting of at least one anti-diabetic drug, an anti-hypertensive drug, and a therapeutically effective amount of at least one orally active anti-inflammatory drug.

In certain preferred embodiments, the pharmaceutical formulation of the invention comprises therapeutically effective amounts of at least two drugs selected from the group consisting of at least one an anti-hypertensive drug, at least one anti-diabetic drug, at least one drug for the treatment of hyperlipidemia, at least one anti-obesity drug, and at least one anti-inflammatory drug. In certain embodiments, the pharmaceutical formulation of the invention comprises therapeutically effective amounts of a drug in at least three, at least four or at least five of the afore-mentioned drug classes. In certain preferred embodiments, the pharmaceutical formulation of the invention comprises therapeutically effective amounts of at least one an anti-hypertensive drug, at least one anti-diabetic drug, at least one anti-obesity drug, and at least one anti-inflammatory drug. In certain preferred embodiments, the pharmaceutical formulation of the invention comprises therapeutically effective amounts of at least one an anti-hypertensive drug, at least one anti-diabetic drug, at least one drug for the treatment of hyperlipidemia, at least one anti-obesity drug, and at least one anti-inflammatory drug.

In certain preferred embodiments, the pharmaceutical formulation of the invention includes an anti-hypertensive drug that is a blocker of Renin-Angiotensin System (RAS).

In certain preferred embodiments in which the metabolic syndrome is Type II diabetes, the pharmaceutical formulation comprises therapeutically effective amounts of a combination of one or more anti-inflammatory drug(s) and at least one drug used in the treatment of Type II Diabetes. In certain of such embodiments, the pharmaceutical formulation further comprises a drug that is a blocker of Renin-Angiotensin System (RAS).

In certain preferred embodiments in which the metabolic syndrome is hyperlipidemia, the pharmaceutical formulation of the invention comprises therapeutically effective amounts of a combination of one or more anti-inflammatory drug(s) and one or more drugs selected from the group consisting of a statin, niacin, a fibrate, and combinations of any of the foregoing.

In certain preferred embodiments in which the metabolic syndrome involves obesity, the pharmaceutical formulation of the invention comprises therapeutically effective amounts of a combination of one or more anti-inflammatory drug(s) and one or more drugs selected from the group consisting of an anti-obesity drug, an anti-diabetic drug, and combinations of any of the foregoing. In certain preferred embodiments, the pharmaceutical formulation for the treatment of obesity further comprises a drug for the treatment of hyperlipidemia and/or hypertension.

In certain preferred embodiments in which the metabolic syndrome involves hypertension, the pharmaceutical formulation of claim 1 comprises therapeutically effective amounts of one or more drugs selected from the group consisting of a beta blocker, a diuretic, an ACE inhibitor, an Angiotension II Receptor Blocker (ARBs), Calcium channel blocker (CCBs), a central agonist, a peripheral-acting adrenergic blocker, a direct vasodilator, a direct renin inhibitor, and combinations of any of the foregoing; and at least one anti-inflammatory drug. In certain preferred embodiments, the pharmaceutical formulation of the invention for the treatment of metabolic syndrome that involves hypertension further comprises therapeutically effective amounts of one or more drugs selected from the group consisting of an anti-obesity drug, an anti-diabetic drug, a drug for the treatment of hyperlipidemia, and combinations of any of the foregoing.

The pharmaceutical formulation may be any that is known to those skilled in the art and useful to administer the drugs in the FDC. In certain preferred embodiments, the pharmaceutical formulation is an oral solid dosage form. In other preferred embodiments, the pharmaceutical formulation is an injectable formulation which is administered parentarally (intravenous, subcutaneous, intramuscular, or intramedullary injection). In yet other embodiments of the invention, the pharmaceutical formulation of is administered transdermally or nasally. The pharmaceutically formulation of claim 1, which is in liquid form.

In certain preferred embodiments which include an antidiabetic drug(s), the anti-inflammatory drug and the antidiabetic drug(s) are in immediate release form. In other preferred embodiments, at least one of the drugs is in controlled or delayed release form.

The invention is further directed in part to a method of treating metabolic syndrome, comprising administering to a human patient having a condition selected from the group consisting of Type II diabetes, Hypertension, Hyperlipidemia and Obesity, a pharmaceutical formulation for the treatment of metabolic syndrome comprising a therapeutically effective amount(s) of a drug selected from the group consisting of at least one anti-diabetic drug; or a therapeutically effective amount of at least one anti-hypertensive drug; or a therapeutically effective amount of at least one anti-obesity drug; or a therapeutically effective amount of at least one drug for the treatment of hyperlipidemia; or a combination of any of foregoing; and a therapeutically effective amount of at least one orally active anti-inflammatory drug. In certain embodiments wherein the patient is diabetic or prediabetic, the method further comprises administering a therapeutically effective amount(s) of a drug selected from the group consisting of at least one anti-diabetic drug, a blocker of Renin-Angiotensin System (RAS), and a combination of the foregoing, together with a therapeutically effective amount of at least one orally active anti-inflammatory drug. In further preferred embodiments wherein the metabolic disease involves hypertension, the method further comprises the administration of one or more of an ACE inhibitor, an angiotension II receptor blocker (ARBs), a DPPIV inhibitor, at least one anti-diabetic drug, a blocker of Renin-Angiotensin System (RAS), together with a therapeutically effective amount of at least one orally active anti-inflammatory drug, to the patient.

It is comtemplated that all of the methods of the present invention encompass the chronic treatment of the metabolic condition(s). Thus, in preferred embodiments, the drugs (e.g., antidiabetic, antihypertensive, anti-obesity, drug for hyperlipidemia; together with an anti-inflammatory drug) are administered to the patient on a chronic basis. In certain preferred embodiments, these drugs are administered as personalized medicines to clinically manage Type II diabetes or prediabetes in stratified groups of human patients based on their cardiometabolic risk factor profiles. In further preferred embodiments, the drugs are administered as personalized medicines to clinically manage Type II diabetes or prediabetes groups of patients stratified for lack of adequate therapeutic efficacy with prior treatments.

In embodiments in which the metabolic syndrome involves Type II diabetes, in certain embodiments the method further comprises concomitantly administering insulin to the patient to achieve better blood glucose control. In certain of such preferred embodiments, dosage is adjusted on the basis of glucose measurements.

The invention is further directed in part to the use of more than one and as many as five orally-active drug FDC of IR, IR and DR and IR and ER formulations for other therapeutic areas.

This invention relates to the pro-inflammatory component of the complex pathophysiology (FIG. 3) as well as the cardiometabolic risk factor clusters (CMRFC) and/or comorbidities of the metabolic syndrome.

This invention relates in part to the Renin-Angiotensin system (RAS) that exists in the pancreatic islets.

This invention describes the overall clinical benefits of blocking the pro-inflammatory components and the RAS in combination with currently marketed drugs that are used to treat Type II diabetes, Hypertension, Hyperlipidemia and Obesity.

This invention describes the overall clinical benefits of blocking the pro-inflammatory components and the RAS in combination with drugs that will be discovered and developed in the future to treat Type II diabetes, Hypertension, Hyperlipidemia and Obesity.

Inflammation and RAS are critical components of the development of insulin resistance which is a pathological hallmark of Type II diabetes as well as the metabolic syndrome. This invention describes customized unique formulation strategies that combine anti-metabolic disease drugs with anti-inflammatory drugs and/or blockers of RAS in the form of Immediate release (IR) or Quick Release (QR) and Extended release (ER)/Sustained release (SR)/Controlled release (CR) and Delayed release (DR) depending on the needs of the individual patient or group of patients.

In certain embodiments of the invention, Fixed Dose combinations (FDC) of drugs may contain blockers or activators of therapeutic targets relevant for treating metabolic diseases and the inflammatory component of the pathophysiology.

In certain embodiments of the invention, FDC formulation of drugs will be prepared in fixed dose ratios to achieve the intended therapeutic efficacy for preventing the incidence of (prophylactic) or delay the progression of prediabetes into insulin resistance syndrome or metabolic syndrome.

In certain embodiments of the invention, FDC of drugs will be prepared in fixed dose ratios to achieve the intended (e.g. anti-type II diabetes, anti-hypertensive, lipid lowering and anti-obesity or a combination of thereof) therapeutic efficacy.

In certain embodiments of the invention, depending on the cardiometabolic risk factor profiles of stratified patients, the FDC of drugs will be prepared in fixed dose ratios of two drug combinations (e.g. anti-inflammatory and anti-type 2 diabetic, anti-inflammatory and lipid lowering or anti-inflammatory and anti-hypertensive or anti-inflammatory and anti-obesity) or three drug combinations (e.g. Anti-inflammatory, Anti-Type II diabetic, Anti-hypertensive or Anti-inflammatory, Anti-Type II diabetic, Lipid lowering or Anti-inflammatory, Anti-Type II diabetic, Anti-obesity or Anti-inflammatory, Anti-hypertensive, Lipid lowering or Anti-inflammatory, Anti-obesity, Anti-hypertensive or Anti-inflammatory, Lipid lowering, Anti-obesity) or four drug combinations (e.g. Anti-inflammatory, Anti-Type II diabetic, Lipid lowering, Anti-hypertensive or Anti-inflammatory, Anti-Type II diabetic, Lipid lowering, Anti-obesity or five drug combinations (e.g. Anti-inflammatory, Anti-Type II diabetic, Lipid lowering, Anti-hypertensive, Anti-obesity) to achieve the intended therapeutic efficacy. In certain embodiments, the FDC of drugs will be prepared in fix dose ratios of more than five drug combinations.

In certain embodiments of the invention, the FDC of drugs will be prepared in fixed dose ratios of two drug combinations of two, three, four and five drug combinations includes an anti-diabetic injectable.

In certain embodiments of the invention, drugs will be formulated with the same drug as FDC of IR and ER.

In certain embodiments of the invention, FDC of drugs will be formulated to contain the same drug in the form of IR and DR.

In certain embodiments of the invention, FDC of drugs will be formulated to contain IR and DR of the same drug along with ER of a second drug.

In certain embodiments of the invention, FDC of drugs will be formulated to contain IR and ER of the same drug along with the IR of a second drug.

In certain embodiments of the invention, FDC of drugs will be formulated to contain IR of one drug, ER of a second and a third drug and IR of a fourth drug.

In certain embodiments of the invention, combination of drugs will be made as a monolithic FDC tablet composed of two or more active formulations mixed and compressed in a single layer tablet.

In certain embodiments of the invention, FDC of drugs will be prepared either in the form of a multi-layered monolithic tablet.

In certain embodiments of the invention, FDC of drugs will be prepared either in the form of a multi-layered monolithic tablet inside a tablet.

In certain embodiments of the invention, FDC of drugs will be prepared either in the form of a core tablet-in-tablet or multi-layered multi-disk tablet (MLMDT) consisting of a tablet core formulation surrounded by a second outer formulation.

In certain embodiments of the invention, FDC of drugs will be prepared in the form of beads inside a capsule. Each bead would represent different color and coating level depending on the kinetics of drug release. For example, some beads would release drugs immediately (IR). Some would beads would release for an extended period of time (ER), while some after a long while (DR).

In certain embodiments of the invention, FDC of drugs will be prepared either in the form of mini tablets inside a capsule. Uniquely color-coded capsules represent customized formulations that contain a unique combination of drugs with predetermined modified release kinetics. Colors and shapes of tablets represent individual drugs (either same or different) and coating level depending on the kinetics of drug release. For example, white colored tablet is released immediately (IR or QR), red colored tablet is released for an extended period of time (ER or SR or XR) and light green tablet is released after a long delay (DR).

In certain embodiments of the invention, FDC of drugs will be prepared in liquid formulations.

In certain embodiments of the invention, the FDC formulation comprises therapeutically effective amounts of a combination of an anti-inflammatory drug(s) and one or more drugs which are used in the treatment of Type II Diabetes drugs. The drugs can be in immediate release form, or they can be in controlled (used synonymously herein with sustained or extended) release form. In yet other embodiments one or more of the drugs can be in immediate release form and the other(s) in controlled or delayed release form. In certain embodiments of the invention, the FDC formulation comprises a combination of an anti-inflammatory drug and two drugs which are used in the treatment of Type II Diabetes. For example, anti-diabetic drugs may be chosen from one or more of a sulfonylurea, a biguanide (e.g., metformin and the like), a DPP-IV inhibitor, an SGLT-2 inhibitor, and/or an incretin mimetic. In preferred embodiments, an anti-inflammatory drug is included in the FDC formulation.

In certain embodiments of the invention, the FDC formulation comprises therapeutically effective amounts a combination including one or more lipid lowering drugs. The drugs can be in immediate release form, or they can be in controlled (used synonymously herein with sustained or extended) release form. In yet other embodiments one or more of the drugs can be in immediate release form and the other(s) in controlled release form. In certain embodiments of the invention, the FDC formulation comprises a combination of at least two of an anti-inflammatory drug, a statin and niacin. In certain other embodiments of the invention, the FDC formulation comprises a combination of at least two of an anti-inflammatory drug, a statin, niacin and a fibrate. In certain other embodiments of the invention, the FDC formulation comprises a combination of at least two of an anti-inflammatory drug, a statin, a fibtrate and niacin. These drugs can be in controlled (used synonymously herein with sustained or extended) release form one or more of the drugs can be in immediate release form and the other(s) in controlled or delayed release form. In preferred embodiments, an anti-inflammatory drug is included in the FDC formulation.

In certain embodiments of the invention, the FDC formulation comprises therapeutically effective amounts a combination including one or more anti-hypertensive drugs. The drugs can be in immediate release form, or they can be in controlled (used synonymously herein with sustained or extended) or delayed release form. In yet other embodiments one or more of the drugs can be in immediate release form and the other(s) in controlled or delayed release form. In certain embodiments of the invention, the FDC formulation comprises a combination of a diuretic and a beta blocker. In certain other embodiments of the invention, the FDC formulation comprises a combination of at least two of an anti-inflammatory drug, a beta blocker, a diuretic, an ACE inhibitor, an Angiotension II Receptor Blocker (ARBs), Calcium channel blocker (CCBs), a central agonist, a peripheral-acting adrenergic blocker, a direct vasodilator, or a direct renin inhibitor. These drugs can be in controlled (used synonymously herein with sustained or extended) or delayed release form and one or more of the drugs can be in immediate release form and the other(s) in controlled release form. In preferred embodiments, an anti-inflammatory drug is included in the FDC formulation.

In certain embodiments of the invention, the FDC formulation comprises therapeutically effective amounts a combination including one or more anti-obesity drugs. The drugs can be in immediate release form, or they can be in controlled (used synonymously herein with sustained or extended) or delayed release form. In yet other embodiments one or more of the drugs can be in immediate release form and the other(s) in controlled or delayed release form. In certain other embodiments of the invention, the FDC formulation comprises a combination of at least two of an anti-inflammatory drug, an anti-obesity drug, a DPPIV inhibitor, and an incretin mimetic. These drugs can be in controlled (used synonymously herein with sustained or extended) or delayed release form and one or more of the drugs can be in immediate release form and the other(s) in controlled release form. In preferred embodiments, an anti-inflammatory drug is included in the FDC formulation.

In certain embodiments of the invention, the FDC formulation comprises therapeutically effective amounts a combination including one or more drugs for prediabetes or the prevention of onset of Type II diabetes. The drugs can be in immediate release form, or they can be in controlled (used synonymously herein with sustained or extended) or delayed release form. In yet other embodiments one or more of the drugs can be in immediate release form and the other(s) in controlled or delayed release form. In certain other embodiments of the invention, the FDC formulation comprises a combination of at least two of an anti-inflammatory drug, an an ACE inhibitor, an angiotension II receptor blocker (ARBs), and an antidiabetic drug as previously described above. These drugs can be in controlled (used synonymously herein with sustained or extended) or delayed release form and one or more of the drugs can be in immediate release form and the other(s) in controlled release form. In certain preferred embodiments, the FDC formulation includes an anti-diabetic in both immediate release and delayed release form (e.g., a biguanide such as metformin). In preferred embodiments, an anti-inflammatory drug is included in the FDC formulation.

In certain embodiments of the invention, the FDC formulation comprises therapeutically effective amounts a combination including one or more drugs for the treatment of metabolic syndrome. The drugs can be in immediate release form, or they can be in controlled (used synonymously herein with sustained or extended) or delayed release form. In yet other embodiments one or more of the drugs can be in immediate release form and the other(s) in controlled or delayed release form. In certain other embodiments of the invention, the FDC formulation comprises a combination of at least two of (and preferably at least three of or all of the following) an anti-inflammatory drug, an ACE inhibitor, an angiotension II receptor blocker (ARBs), and a DPPIV inhibitor. These drugs can be in controlled (used synonymously herein with sustained or extended) or delayed release form and one or more of the drugs can be in immediate release form and the other(s) in controlled release form. In preferred embodiments, an anti-inflammatory drug is included in the FDC formulation.

In certain embodiments of the invention, the FDC formulation comprises therapeutically effective amounts of Type II diabetes drugs such as older shorter-acting sulfonyl ureas such as Dymelor (acetohexamide, 50 mg to 500 mg), Diabenese (chloropropamide, 100 mg to 500 mg), Orininase (tolbutamide, 250 to 500 mg), Tolinase (Tolazamide, 100 to 250 mg) or newer sulfonyl ureas such as Glucotrol (Glipizide, 2.5 mg to 30 mg), Glucotrol XL (2.5 mg to 100 mg), DiaBeta (glyburide, 1.25 mg to 20 mg), Micronase (1.25 mg to 20 mg), Glynase (glyburide/Glibenclamide, 0.75 mg to 12 mg), Glynase PresTab (glyburide, 0.75 mg to 12 mg), Amaryl (glimepiride, 1 mg to 8 mg) or biguanides such as Metformin (Glucophage, Glucophage XR, Riomet, Fortamet and Glumetza, 250 mg to 2550 mg) or Thiozolidinediones (TZDs) such as Actos (pioglitazone, 5 mg to 30 mg) Avandia (rosiglitazone, 2 mg to 8 mg), or Alpha glucosicase inhibitors such as Precose (acarbose, 25 mg to 100 mg), Glyset (miglitol, 25 mg to 100 mg) or Dipeptidyl peptidase inhibitors (DPPIV) such as Januvia (sitagliptin, 10 mg to 100 mg), Nesina (alogliptin, 2.5 mg to 25 mg), Onglyza (linagliptin, 2.5 mg) or incretin mimetic such as Repaglinide (Prandin, 2.5 ug to 10 ug), Nateglinide (Starlix, 2.5 ug to 10 ug), Exenatide (Byetta, Bydureon, 2.5 ug to 10 ug), Liraglutide (Victoza, 0.25 mg to 1.8 mg) or Sodium Glucose co-transporters (SGLT-2) inhibitors such as Farxiga (dapagliflozin, 2.5 mg to 10 mg) and Invokana (canagliflozin, 50 mg to 100 mg).

In certain embodiments of the invention, the FDC formulation comprises therapeutically effective amounts of high cholesterol/lipid lowering drugs such as Atorvastatin (Lipitor, 5 mg to 80 mg), Fluvastatin (Lescol, 10 mg to 80 mg), Lovastatin (Mevacor, 5 mg to 80 mg), Provastatin (Pravachol, 20 mg to 80 mg), Rosuvastatin (Crestor, 2.5 mg to 40 mg), Simvastatin (Zocor, 2.5 mg to 40 mg) or Nicotinic acid such as Nicolar (250 mg to 500 mg), Niaspan (250 mg to 2000 mg) or Bile acid resins (sequestrants) such as Questran (4.5 g to 9 g) and Questran Light (2.5 g to 24 g), Colestid (1 g to 16 g), Welchol (1 g to 3.75 g) or Fibrates such as Atromid (20 mg to 145 mg), Tricor (24 mg to 145 mg), Lopid (500 mg to 1200 mg), Ezetimide (Zetia, 2.5 mg to 10 mg).

In certain embodiments of the invention, the FDC formulation comprises therapeutically effective amounts of anti-inflammatory drugs such as Celebrex (Celecoxib, 25 mg to 200 mg), Advil, Motrin (Ibuprofen, 100 mg to 3200 mg), Aleve (Naproxen, sodium, 100 mg 1650 mg), Ascriptin (200 mg to 325 mg), Ecotrin (50 mg to 325 mg), Asprin (81 mg to 325 mg), Anaprox (Naproxen Sodium, 100 mg to 1650 mg), Clinoril (Sulindac, 100 mg to 400 mg), DayProv (Oxaprozin, 500 mg to 1200 mg), Disalcid (Salsalate, 500 mg to 3 g), Dolobid (Difflumisal, 250 mg to 750 mg), Feldene (Piroxieum, 5 mg to 20 mg), Indocin (Indomethacin, 25 mg to 100 mg), Lodine (Etodolac, 100 mg to 500 mg), Mobic (Meloxicam, 5 mg to 20 mg), Naprosyn (Naproxen, 100 mg to 1650 mg), Relafen (Nabumetone, 500 mg to 2000 mg), Toradol (Ketorolactromethamine, 10 to 40 mg), Vimovo (Naproxen/esomeprazole, 375 mg/20 mg), Voltaren (Diclofenac, 50 mg 100 mg), Xeljanz (Tofactinib Citrate, 2.5 mg to 5 mg), Anakinra (Anti-IL6 Ab, 50 mg to 100 mg), Humira (Anti-TNF-alpha Ab, 5 mg 10 mg).

In certain embodiments of the invention, the FDC formulation comprises therapeutically effective amounts of anti-hypertensive drugs such as Diuretics (Aldactone (Spiranolactone, 25 mg to 400 mg), Dyrenium (Triampeterene, 50 mg to 100 mg), Esidix, Hydrodiuril & Microzide (Hydrochlorothiazide or HCTZ, 12.5 mg to 100 mg), Hygroton & Thalitone (Chlorthalidone, 10 mg to 100 mg), Lasix (Furosemide, 10 mg to 600 mg), Lozol (Amiloride hydrochloride, 1.25 mg to 5 mg), Mykrox (0.5 mg) and Zaroxylyn (2.5 mg) (Metolazone) or Beta blockers (Blocadren (Timolol, 5 mg to 60 mg), Cartrol (Cartelol Hydrochloride, 1.25 mg to 10 mg), Coreg (Corredilol, 3.125 mg to 50 mg), Coregard (Nadolol, 20 mg to 160 mg), Inderal (Propranolol, 20 mg 640 mg), Kerlone (Betaxolol, 5 mg to 40 mg), Levatol (Penbutolol Sulfate, 10 mg to 80 mg), Lopressor & Toprol XL (Metoprolol, 50 mg to 450 mg), Sectral (Acebutolol, 200 mg to 1200 mg), Tenormin (Atenolol, 25 mg to 200 mg), Visken (Pindolol, 2.5 mg 60 mg), Zebeta (Bisprolol fumarate, 1.25 mg to 20 mg), Normodyne and Trandate (Labetolol, 50 mg to 2400 mg) or Alpha blockers (Cardura (Doxazocin, 1 mg to 16 mg), Hytrin (Terazocin, 1 mg to 20 mg), Minipress (Prazocin, 1 mg to 20 mg) or Angiotensin-converting enzyme inhibitors: Accupril (Quinapril, 5 mg to 80 mg), Altace (Ramipril, 2.5 mg to 20 mg), Capoten (Captopril, 25 mg to 450 mg), Mavik (Trandolapril, 1 mg to 8 mg), Lotensin (Benazepril, 5 mg to 40 mg), Monopril (Fosinopril, 5 mg to 80 mg), Prinvil & Zestril (Lisinopril, 5 mg to 80 mg), Univasc (moexipril, 5 mg to 60 mg), Vasotec (Enalapril, 2.5 mg to 40 mg) or Angiotensin receptor blockers: Atacand (Candesartan, 4 mg to 32 mg), Avapro (Irbesartan, 75 mg to 300 mg), Benicar (Olmesartan, 10 mg to 40 mg), Cozaar (Losartan, 25 mg to 100 mg), Diovan (Valsartan, 40 mg to 320 mg), Micardis (Telmisartan, 20 mg to 80 mg), Teveten (Eprosartan, 400 mg to 800 mg) or Calcium channel blockers, Adalat and Procardia (Nifedipine, 10 mg to 60 mg), Calan, Covera, Isoptin, Verelan, and others (Verapamil, 120 mg to 300 mg), Cardene (Nicardipine, 20 mg to 60 mg), Cardizem Cartia, Dilacor, and Tiazac (Diltiazem, 5 mg to 420 mg), DynaCirc (Isradipine, 2.5 mg to 10 mg), Plendil (Felodipine, 2.5 mg to 10 mg), Norvasc (Amlodipine, 2.5 mg to 10 mg), Sular (Nisoldipine, 5 mg to 60 mg) or Central agonists: Aldomet (Methyldopa, 250 to 500 mg), Catapres (Clonidine, 0.1 mg to 2.4 mg), Tenex (Guanfacine, 1 mg to 3 mg), Wytensin (Guanabenz, 2 mg to 32 mg) or Peripheral-acting adrenergic blockers: Hylorel (Guanadrel, 5 mg to 75 mg), Ismelin (Guanethidine, 5 mg to 50 mg), Serpasil (Reserpine, 0.1 mg to 0.5 mg) or Direct vasodilators: Loniten (Minoxidil, 2.5 mg to 100 mg), Apresoline (Hydralazine, 5 mg to 300 mg) or Direct renin inhibitors (Tekturana (Aliskiren, 75 mg to 300 mg).

In certain embodiments of the invention, the FDC formulation comprises therapeutically effective amounts of Anti-Obesity drugs such as Orlistat (Xenical, 60 mg to 120 mg), Orlistat (Alli) OTC (30 mg to 60 mg), Phentermine (15 mg to 37.5 mg) Lorcaserin (Belviq, 5 mg to 10 mg), Phentermine/Topimerate (Qsymia, 3.75 mg/23 mg to 7.5 mg/46 mg).

For purposes of the present invention, it is to be understood that the above dosages of drugs are approximate and that the appended claims encompass obvious variations of the same. It is further contemplated that other drugs in the same drug classes can be substituted for the above drugs/dosages, and such obvious changes are encompassed by the appended claims.

For purposes of the present invention, the term “drug” is used interchangeably with the term “agent” or “active agent”.

For purposes of the present invention, the term “FDC” or “fixed dose combination” refers to a pharmaceutical formulation containing a specific therapeutically effective dose of one or more orally-active anti-inflammatory drugs as described in this specification and a specific therapeutically effective dose of one or more anti-metabolic disease drugs as described in this specification in any pharmaceutically acceptable carrier(s) such that the pharmaceutical formulation is suitable, e.g., for oral administration, parenteral administration, transdermal administration, nasal administration, buccal administration, topical administration. It is contemplated that the FDC may comprise one or more dosage forms administered concurrently in order to provide the desired therapeutic effect. It is further contemplated that the FDC may encompass one or more routes of administration of the anti-inflammatory and anti-metabolic disease drugs as described herein in order to obtain a desired therapeutic effect. It is contemplated that multiple FDC combinations of the same drug combinations may be made available in order to provide a desired therapeutic effect in different human patients.

### BRIEF DESCRIPTION OF FIGURES

FIG. 1 is comparison of white adipose tissue (WAT) from a lean animal with that of an obese insulin resistant animal: infiltration of macrophages into WAT.

FIG. 2 is a schematic illustration of the Complex pathophysiology of metabolic syndrome.

FIG. 3 shows that deletion of Cox-2 in C57BL/6J obese mice reduces blood glucose levels.

FIG. 4 shows that selective Cox-2 inhibitor, SC58236 reduces HbA1c levels in C57BL/6J obese mice.

FIG. 5 shows that selective Cox-2 inhibitor, SC58236 improves glucose tolerance and elevates plasma insulin levels in C57BL/6J obese mice.

FIG. 6 shows that selective Cox-2 inhibitor, Celecoxib reverses steatohepatitis and inflammation in Wistar rat NASH model.

FIG. 7 shows that metformin improves glucose tolerance in C57BL/6J diet-induced obesity (DIO) mice.

FIG. 8 shows that Valsartan improves glucose tolerance in C57BL/6J diet-induced obesity (DIO) mice.

### DETAILED DESCRIPTION OF THE INVENTION

Inflammation and RAS play an important pivotal role in Type II diabetes and its comorbidities.

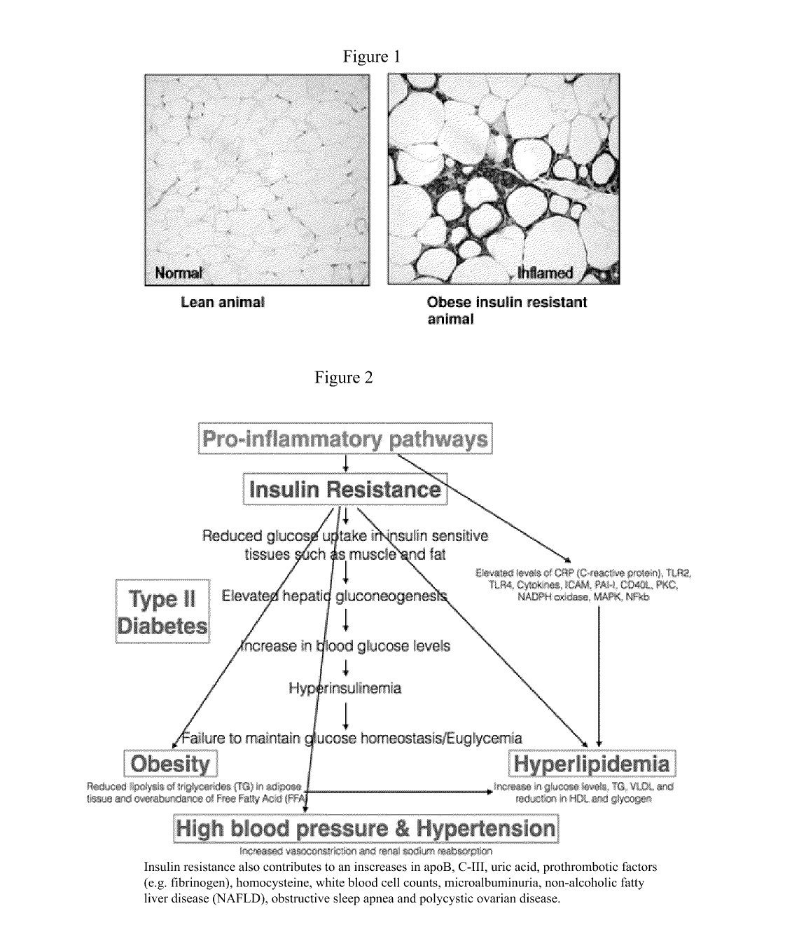

Obesity in general and obesity-triggered low-grade or sub-acute chronic inflammation is the major contributing factor for systemic insulin resistance, systemic glucose intolerance and systemic lipotoxicity (Xu, H. 2013). Extensive signaling cross-talk among inflammatory signaling pathways, innate and adaptive immune systems, glucose metabolic pathways and lipid metabolism pathways in a temporal as well as contextual manner initiate and sustain impaired glucose homeostasis (Shu, C. J. et al. 2012). The events that initiate and maintain inflammation is central to the complex pathophysiology of obesity-triggered insulin resistance include the following. As the primary tissue for storage of fat, white adipose tissue (WAT) undergoes hyperplasia as preadipocytes differentiate into adipocytes and maintains an anti-inflammatory state acting as a sink with a buffering capacity for fat intake and fat influx (Tateya, S. et al. 2013). The buffering capacity of WAT prevents ectopic fat accumulation and consequently from lipotoxicity (Saltiel, A. 2000). The resident M2 macrophages of hyperplasic WAT secrete anti-inflammatory cytokines such as IL-10, IL-4 and IL-13. The buffering capacity for fat intake in obese patients without insulin resistance occurs as a result of suppression of release of non-esterified fatty acids, decrease in the activity of HSL (Hormone-sensitive lipase) and ATGL (Adipose triglyceride Lipase) (Mitrou, P. et al. 2013). Hyperplasic WAT also have the capacity for increased clearance of triacyl glycerol due to increase in the activity of Lipoprotein lipase (LPL). As white adipocytes accumulate more triacyl glycerol as a result of longterm nutrient excess, they become hypertorphic by increasing in size with concomitant release of monocyte chemoattractant protein (MCP-1) which creates a gradience for recruitment and infiltration of circulating pro-inflammatory M1 macrophages into WAT as shown in FIG. 2 by immunostaining of sections of WAT with macrophagee surface marker F4/80 (Coenen, K. R. et al. 2007). Infiltrated M1 macrophages are retained in WAT due to the synthesis of a guidance molecule known as Netrin-1. Deletion of Netrin-1 results in emigration of infiltrated macrophages in a diet-induced obesity mouse model and improves insulin sensitivity (Ramkhelawon, B. et al. 2014). As the number of M1 macrophages increase, the WAT gradually shifts from an anti-inflammatory state into a pro-inflammatory state as a result of secretion of pro-inflammatory cytokines and chemokines such as IL-6, TNF-alpha, IL-1beta, IL-18, CCL2, CCL3, CXCL8, C-reactive protein (CRP) (Quatanami, M. and Lazar, M. A. 2007). WAT gradually loses its inherent buffering capacity for fat intake and it shifts to a pro-inflammatory state. Elevation in the levels of pro-inflammatory cytokines increases activity of JNK-1 which phosphorylates serine residues on IRS-1 (Insulin receptor substrate-1) rendering it incapable of triggering cascade of signaling events further downstream from IRS-1 in insulin sensitive tissues such as adipocytes, skeletal muscle and hepatocytes. Gradual development of systemic resistance contributes to systemic glucose intolerance as a result of a number of events including: decrease in the levels of adiponectin, Glut4 and IL-10, activation of toll-like receptor, TLR-4 as well as activation of the receptor for Palmitic acid, UNC5b, increase in the levels of leptin, resistin, visfatin and Netrin-1, increase in the levels of PGE metabolites as well as an increase in the level of oxidative stress parameters, pancreatic beta cell dysfunction, activation of SOC (1,2,3)-mediated phosphorylation of IRS-1 and IRS-2, increase in the degradation of IRS-1 and IRS-2, decrease in the levels of thermogenic genes, PGC-1 and UCP-1, and an increase in NLRP3 inflammasome, caspase-1 and ASC (Wang, X. et al. 2014, Boucher, J. et al. 2014). Development of insulin resistance contributes to a decrease in the amount of intracellular glucose which results in elevated levels of blood glucose levels and an increase in the release of non-esterified fatty acid from WAT which results in elevation of circulating levels of free fatty acids (FFA) and triglycerides (TG). It is an established fact that inflammation is central to the complex pathophysiology of not only T2DM and it is also central to the etiology of diabetes-related complications. As a result of systemic glucose intolerance, Type 2 diabetes patients develop systemic glucolipotoxicity.

Infiltration of circulating M1 macrophages in to the White adipose tissue (WAT) is one of the early events in shifting the WAT from an anti-inflammatory state to an inflammatory state. Comparison of WAT from a lean animal with that of an obese animal is shown in FIG. 2. Sections of WAT are imuunostained with a macrophage-specific marker F4/80 (Coenen, K. R. et a. 2007). Macrophages are shown in red.

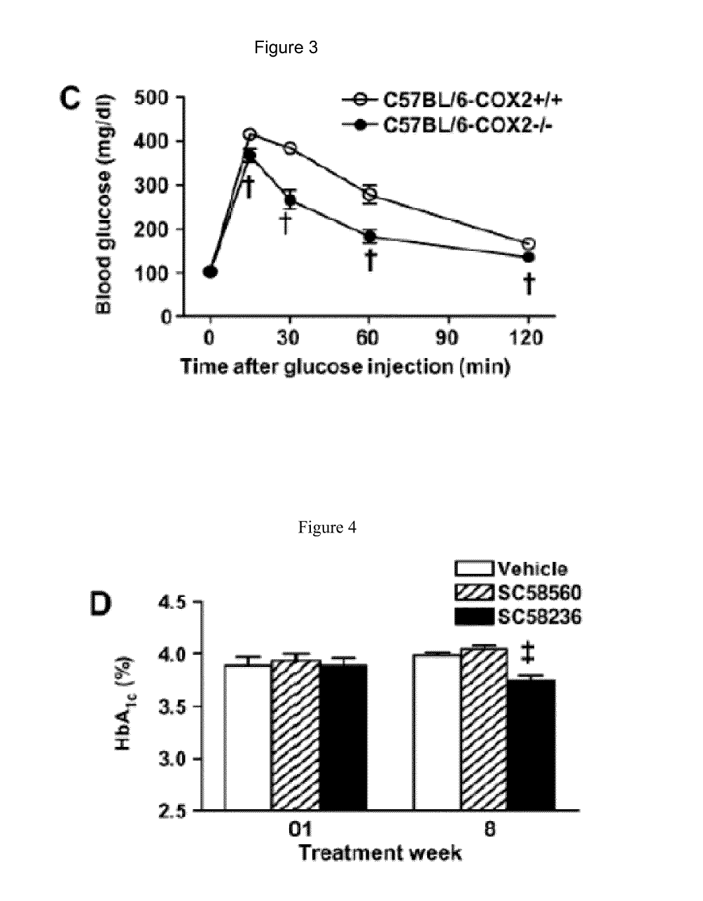

Inflammation is the underlying cause of the pathophysiology of not only insulin resistance but also a critical contributing factor to the comorbidities such as hypertension, dyslipidemia and obesity. The complex pathophysiology of the metabolic syndrome is illustrated in FIG. 3.

Activation of inducible Cyclooxygenase, Cox-2 or activation of constitutive Cox-2 plays a critically important role in the initiation of obesity-triggered inflammation. A link between elevation of blood glucose levels and activation of Cox-2 in pancreatic beta cells is well established (Oshima, H. et al. 2006). C57BL/6J DIO (Diet-induced obesity) mice are an established polygenic preclinical translational model for Type II diabetes, prediabetes, glucose intolerance and insulin resistance. Selective deletion of Cox-2 improves glucose tolerance after glucose loading (2 g/Kg) after fasting mice for 6 hours in an intraperitoneal glucose tolerance test (IPGTT) as shown FIG. 4 (Fujita, H. et al. 2007). Blood samples were taken from the cavernous sinus by capillary under anesthetized condition at 0.5 h before (0 h in the figure) and at 0.5, 1, 2 and 5 h after glucose loading. Blood glucose was measured using Glucord Diameter. (Arkray, Tokyo, Japan).

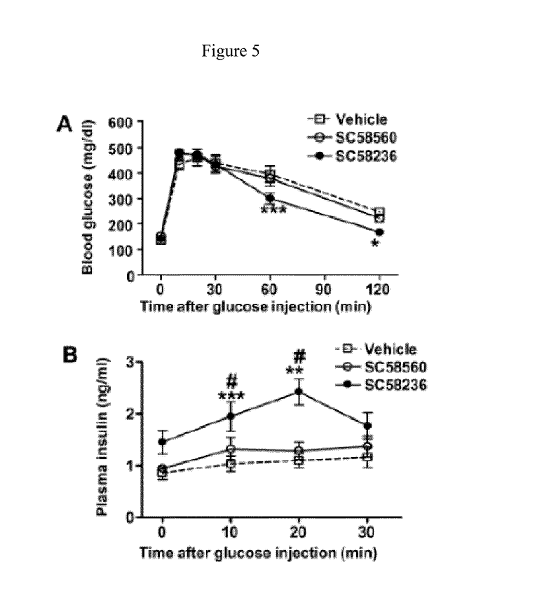

Cox-2 is the predominant species of cyclooxygenase expressed pancreatic beta islet cells. Excessive nutrient intake as well as activation of RAS results in the activation of Cox-2. Cox-2-mediated elevation in PGE2 impairs pancreatic compensation and leads to islet cell dysfunction (Poitout, V and Robertson, R. P. 2008). Elevation in PGE2 levels contributes to reduction beta islet cell mass due to decreased hyperplasia as well as due to apoptosis of the beta islet cells. As shown in FIG. 5, selective Cox-2 inhibition reduces HbA1c levels by about 0.4%. SC58560 and SC58236 are selective Cox-1 and Cox-2 inhibitors respectively (Fujita, H. et al. 2007). HbA1c levels were determined using a DCA 2000 Analyzer (Bayer, Elkhart, Ind.).

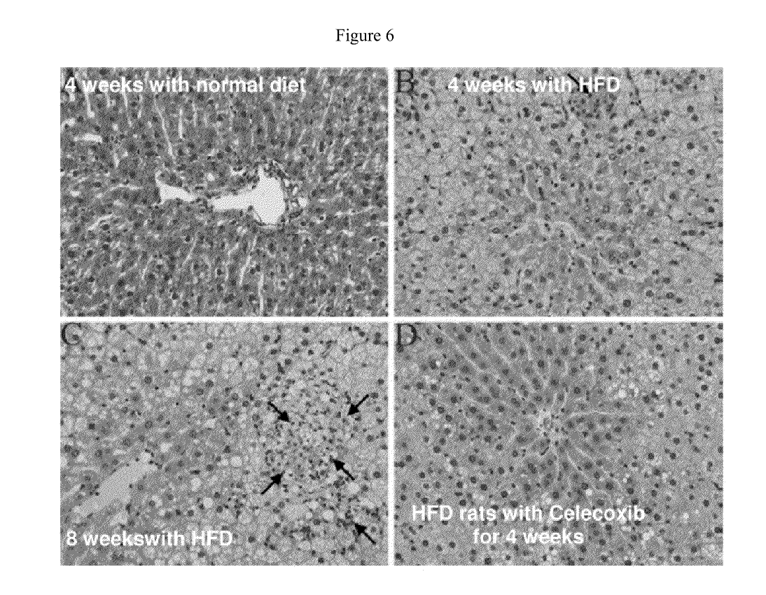

Selective inhibition of Cox-2 improves glucose tolerance in an intraperitoneal glucose tolerance test (IPGTT) and elevates plasma insulin levels as shown in FIG. 6 (Fujita, H. et al. 2007). IPGTT was performed after mice were fasted for 6 hours. After glucose loading (2 g/Kg), blood samples were taken from the cavernous sinus by capillary under anesthetized condition at 0.5 h before (0 h in the figure) and at 0.5, 1, 2 and 5 h. Blood glucose was measured using Glucord Diameter. (Arkray, Tokyo, Japan). Plasma insulin levels after glucose injection were measured using a commercial insulin ELISA kit (Morinaga, Yokohama, Japan).

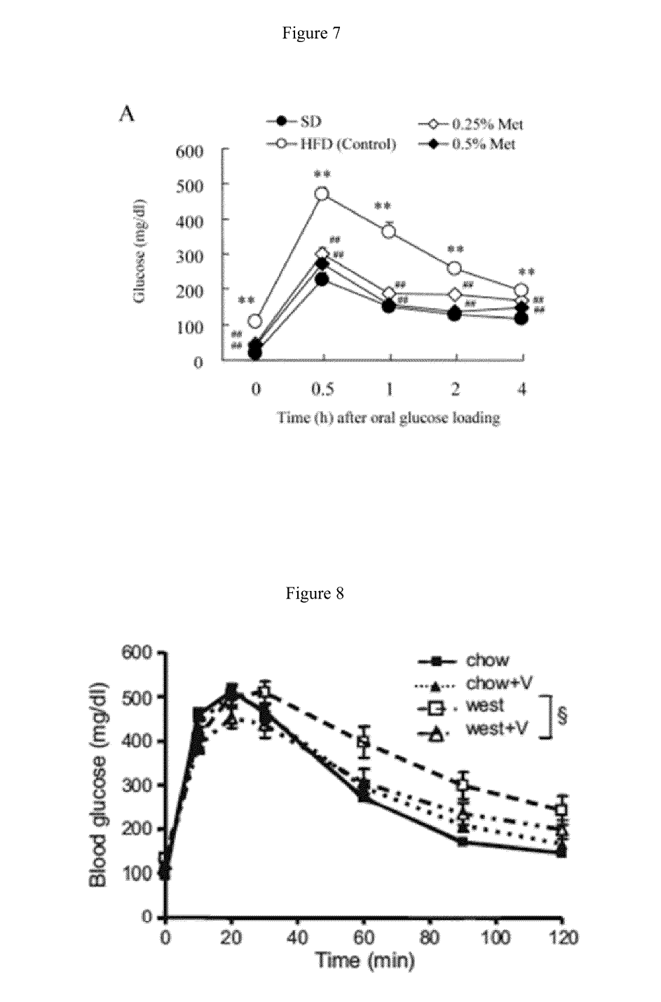

Non-alcoholic staeatohepatitis (NASH) is a condition that coexists with Type 2 diabetes. Celecoxib, a selective Cox-2 inhibitor reverses steatohepatitis and inflammation in Wistar rat NASH model (Chen, J. et al. 2011). FIG. 7 shows the histological changes in the liver of rats on HFD (high fat diet) and Celecoxib-treated rats. Rats on normal diet were used as controls. A. Wistar rats n normal diet for 4 weeks showed normal liver histology, (B) When rats fed the HFD for 4 weeks, fat droplets and small groups of inflammatory cells (arrow) were observed. (C) After 8 weeks of HFD feeding, severity of steatohepatitis greatly increased. (D) Treatment of HFD rats with Celecoxib (20 mg/kg/day) for 4 weeks attenuated steatohepatitis. Arrows point to fat droplets and groups of inflammatory cells. Sections of liver were stained with eosin and hematoxylin.

Metformin, a biguanide is the gold standard among the currently marketed Type II diabetes drugs. As shown in FIG. 8, it improves glucose tolerance in C57BL/6J diet-induced obese (DIO) mice model in oral glucose tolerance test (OGTT) in which the mice were challenged with 2 g/Kg glucose after fasting mice for 20 hours (Matsui, Y. et al. 2010). 0.25% and 0.5% Metformin are 150 mg/Kg/day and 300 mg/Kg/day respectively. Plasma glucose levels were determined by using Glucose II-Test Wako purchased from Wako Pure Chemical Industries, Ltd. (Osaka, Japan).

Valsartan (Diovan) is a selective Angiotensin receptor Type I receptor blocker (ARB). RAS play a critically important role in inflammation-indued beta islet cell dysfunction as well in the process of pancreatic decompensation. Valsartan has been shown to delay the onset of Type II diabetes in prediabetes patients as well (Andraws, R. and D. L. Brown, 2007). In a randomized controlled, double blind, two-center clinical study conducted for 26 weeks, Valsartan improved beta cells function and insulin sensitivity in subjects with impaired glucose metabolism (Van der Zijl, N. et al. 2011). As shown in FIG. 9, Valsartan improves glucose tolerance in C57BL/6J diet-induced obese (DIO) mice. For testing glucose tolerance, mice were fasted overnight and then injected intraperitoneally (IP) with 2 g/Kg glucose. Blood glucose measurements were performed with an UltraTouch glucometer using blood samples taken from cut tail tips at baseline, 15, 30, 45 and 60 minutes after the injection of glucose (Cole, B. K. et al. 2010).

Methods and processes involved in the preparation of tablets, capsules and tablets inside a capsule will be improved and optimized for the FDC formulations described and disclosed in this application. For example, steps involved in the preparation of a bilayer tablet is schematically illustrated in FIG. 10. Dosing of granules for the bottom layer will be placed in the rotary die from the first hopper followed by recompression of the first layer by the first roller. The first layer will be reduced to a smaller size to create space required for the second feeder. Dosing of the top layer would occur from the second hopper to the rotary die. The filled die will be transferred to the second roller for compression. Final compression of the two layers would result in two distinct layers. Multilayer tablet machines will be equipped with suction nozzles or dust extractor to remove fine powder granules to eliminate cross-contamination between the two layers.

A therapeutic dose of an anti-inflammatory drug will be combined with therapeutic doses of one or more anti-type II diabetic, anti-hypertensive, lipid lowering and anti-obesity drugs with predetermined modified release kinetics to achieve therapeutic as well as kinetic synergies.

Therapeutic doses of a combination of anti-inflammatory drugs, anti-type II diabetes drugs, lipid lowering drugs and anti-obesity drugs will be formulated in the form of fixed dose combination products (FDC) to treat Type II diabetes patients who are stratified into groups based on their cardiometabolic risk factor profiles.

Therapeutic doses of a combination of anti-inflammatory drugs, anti-type II diabetes drugs, lipid lowering drugs and anti-obesity drugs will be formulated in the form of fixed dose combination products (FDC) to treat Type II diabetes patients who are stratified into groups for lack adequate therapeutic efficacy and over all clinical benefit from the prior treatments.

Therapeutic doses of a combination of anti-inflammatory drugs, anti-type II diabetes drugs, lipid lowering drugs and anti-obesity drugs will be formulated in the form of fixed dose combination products (FDC) to prevent or delay the onset of Type II diabetes in Prediabetes patients.

FDC formulations contain the following combinations in the form of single layer monolithic tablet or multi-layered monolithic tablet or in the form of a core tablet-in-tablet or multi-layered multi-disk tablet or beads inside a capsule or tablets inside a capsule but not limited to: (a) therapeutically efficacious Fixed Dose Combinations (FDC) of IR formulations of different drugs; (b) Therapeutically efficacious FDC of IR and ER formulations of different drugs; Therapeutically efficacious FDC of IR and ER formulations of the same drug in combinations with ER formulations of one or more of different drugs; Therapeutically efficacious FDC of IR and ER formulations of the same drug in combinations with ER formulations of one or more of the same drug; and/or Therapeutically efficacious FDC of IR and ER formulations of the same drug in combinations with ER formulations of one or more of different drugs to compensate for potential adverse side effects such as hypoglycemia.

### Preparation of FDC Formulations

Methods and compositions to treat metabolic and cardiovascular diseases have been described in general in several issued patents and patent applications such as U.S. Pat. Nos. 5,561,165, 8,431,522, 7,632,818, 8,586,607, 8,759,334, 8,586,529, 6,846,800, 8,507,451, 5,190,970, 8,586,069 B2, 8,435,550 B2, WO 2008014471 A1, U.S. Pat. No. 8,367,418 B2, WO 2004017896 A2, U.S. Pat. No. 8,008,328 B2, WO 2010088375 A2, US 20130261092 A1, US 20140037739 A1, WO 2005009412 A1, EP 2727587 A1 and WO 2011 078993.

Tablets of the invention described here can be prepared by methods well known in the art. Various methods for IR, QR, ER, SR, XR, DR layers and the vehicles therein are well known in the art. Generally recognized compendium of methods include: Remington: The Science and Practice of Pharmacy, Alfonso R. Gennaro, Editor, 20th Edition, Lippinscott Williams & Wilkins, Philadelphia, Pa.; Sheth et al. (1980) Compressed tablets, in Pharmaceutical dosage forms, Vol 1, edited by Lieberman and Lachtman, Dekker, N.Y.

Published methods (Shende, P. et al. 2012; Khan, Z. et al. 2013) will be optimized further to generate delivery systems in the form of FDC formulations.

For oral, buccal, and sublingual administration, powders, suspensions, granules, tablets, pills, capsules, gelcaps, and caplets are acceptable as solid dosage forms.

These can be prepared, for example, by mixing one or more compounds of the instant present technology, or pharmaceutically acceptable salts or tautomers thereof, with at least one additive such as a starch or other additive. Suitable additives are sucrose, lactose, cellulose sugar, mannitol, maltitol, dextran, starch, agar, alginates, chitins, chitosans, pectins, tragacanth gum, gum arabic, gelatins, collagens, casein, albumin, synthetic or semi-synthetic polymers or glycerides. Optionally, oral dosage forms can contain other ingredients to aid in administration, such as an inactive diluent, or lubricants such as magnesium stearate, or preservatives such as paraben or sorbic acid, or anti-oxidants such as ascorbic acid, tocopherol or cysteine, a disintegrating agent, binders, thickeners, buffers, sweeteners, flavoring agents or perfuming agents. Tablets and pills may be further treated with suitable coating materials known in the art.

Liquid dosage forms for oral administration may be in the form of pharmaceutically acceptable emulsions, syrups, elixirs, suspensions, and solutions, which may contain an inactive diluent, such as water. Pharmaceutical formulations and medicaments may be prepared as liquid suspensions or solutions using a sterile liquid, such as, but not limited to, an oil, water, an alcohol, and combinations of these pharmaceutically suitable surfactants, suspending agents, emulsifying agents, may be added for oral or parenteral administration. As noted above, suspensions may include oils. Such oils include, but are not limited to, peanut oil, sesame oil, cottonseed oil, corn oil and olive oil. Suspension preparation may also contain esters of fatty acids such as ethyl oleate, isopropyl myristate, fatty acid glycerides and acetylated fatty acid glycerides. Suspension formulations may include alcohols, such as, but not limited to, ethanol, isopropyl alcohol, hexadecyl alcohol, glycerol and propylene glycol. Ethers, such as but not limited to, poly(ethyleneglycol), petroleum hydrocarbons such as mineral oil and petrolatum; and water may also be used in suspension formulations.

Immediate release formulations will be prepared by combining super disintegrant such as Croscarmellose sodium and different grades of microcrystalline cellulose in different ratios. To aid disintegration, sodium starch glycolate will added. A formulation method which would result in tablet disintegration of >98% in less than a minute will be used in the final formulation.

Published methods will be used to prepare FDC of drugs either in the form of a form of beads inside a capsule. Each bead would represent different color and coating level depending on the kinetics of drug release. For example, some beads would release drugs immediately (IR). Some would beads would release for an extended period of time (ER), while some after a long while (DR).

As an example, extended release multi-layered matrix tablets will be prepared by using FDC of different drugs and hydrophilic polymer ratio with guar gum, hydroxypropylmethyl cellulose, and xanthan gum as matrix formers. All lubricated formulations will be compressed by wet granulation method.

Multilayer tablet delivery procedure used in the GeoMatrix™ Technology will be used. It consists of a hydrophilic matrix core, containing the active ingredient, and one or two impermeable or semi-permeable polymeric coatings. This technology uses films or compressed polymeric barrier coatings on one or both sides of the core.

The presence of polymeric coatings in the GeoMatrix™ Technology, modifies the hydration/swelling rates of the core and reduces the surface area available for drug release. These partial coatings provide a modulation of the drug dissolution profile: they reduce the release rate from the device and shift the typical time-dependent release rate towards constant release. This technology enables customized levels of controlled release of specific drugs and/or simultaneous release of two different drugs at different rates can be achieved from a single tablet. The combination of layers, each with different rates of swelling, gelling and erosion, is used for the rate of drug release in the body. Exposure of the multilayer tablet as a result of partial coating may affect the release and erosion rates, therefore, transformation of multilayered tablet with exposure on all sides to the gastrointestinal fluids upon detachment of the barrier layer will be considered.

Multi-layered tablets containing combinations of immediate release and modified/extended release of two different drugs or dual release rate of the same drug in a single dosage form will be prepared by using hydrophilic and hydrophobic polymer matrices.

Dual release repeat action multi-layered tablets will be prepared with an outer compression layer with an initial dose of rapidly disintegrating matrix in the stomach and a core inner layer tablet formulated with components that are insoluble in the gastric media but release efficiently in the intestinal environment.

Cross-contamination between different layers will be prevented by using barrier layers of inert/non-functional material.

Quality control methods and best industry practices will be developed and adopted to meet international Pharmacopeial standards.

Multilayered matrix tablets of a combination of immediate and/or extended release tablets will be evaluated for a number of physico-chemical properties (Khan, Z. et al. 2013)

a. Uniformity of weight
b. Content of active ingredient/drug content
c. Friability
d. Hardness
e. Thickness
f. Weight uniformity
g. Disintegration time
h. In vitro drug release/dissolution
i. Stability

Compositions containing the active agent(s) of the present invention can be formulated for administration to a subject via any conventional means including, but not limited to, oral, parenteral (e.g., intravenous, subcutaneous, or intramuscular), buccal, intranasal or transdermal administration routes.

Moreover, the pharmaceutical compositions described herein can be formulated into any suitable dosage form, including but not limited to, aqueous oral dispersions, aqueous oral suspensions, solid dosage forms including oral solid dosage forms, aerosols, controlled release formulations, fast melt formulations, effervescent formulations, self-emulsifying dispersions, solid solutions, liposomal dispersions, lyophilized formulations, tablets, capsules, pills, powders, delayed release formulations, immediate release formulations, modified release formulations, extended release formulations, pulsatile release formulations, multiparticulate formulations, and mixed immediate release and controlled release formulations. In some embodiments, the active agent(s) of the present invention formulations provide a therapeutically effective amount of the active agent(s) of the present invention over an interval of about 30 minutes to about 8 hours after administration, enabling, for example, once-a-day, twice-a-day (b.i.d.), or three times a day (t.i.d.) administration if desired. In one embodiment, the active agent(s) of the present invention particles are formulated into a controlled release or pulsatile solid dosage form for b.i.d. administration. In other embodiments, the active agent(s) of the present invention particles are dispersed in an aqueous dispersion for b.i.d. administration. Generally speaking, one will desire to administer an amount of the active agent(s) of the present invention that is effective to achieve a plasma level commensurate with the concentrations found to be effective in vivo for a period of time effective to elicit a desired therapeutic effect.

The various release dosage formulations discussed above can be characterized by their disintegration profile. A profile is characterized by the test conditions selected. Thus the disintegration profile can be generated at a pre-selected apparatus type, shaft speed, temperature, volume, and pH of the dispersion media. Several disintegration profiles can be obtained. For example, a first disintegration profile can be measured at a pH level approximating that of the stomach (about pH 1.2); a second disintegration profile can be measured at a pH level approximating that of one point in the intestine or several pH levels approximating multiple points in the intestine (about 6.0 to about 7.5, more specifically, about 6.5 to 7.0). Another disintegration profile can be measured using distilled water. The release of formulations may also be characterized by their pharmacokinetic parameters, for example, Cmax, Tmax, and AUC (0-τ).

In one embodiment, the dosage form is a solid oral dosage form which is an immediate release dosage form whereby >80% of the active agent(s) of the present invention particles hours after administration. In other embodiments, the invention provides an (e.g., solid oral) dosage form that is a controlled release or pulsatile release dosage form. In such instances, the release may be, e.g., 30 to 60% of the active agent(s) of the present invention particles by weight are released from the dosage form within about 2 hours after administration and about 90% by weight of the active agent(s) of the present invention released from the dosage form within about 7 hours after administration. In yet other embodiments, the dosage form includes at least one active agent in immediate release form and at least one active agent in delayed release form, or sustained release form. In yet other embodiments, the dosage form includes at least two active agents which are released at different rates as determined by in-vitro dissolution testing or via oral administration.

In some embodiments, the solid dosage forms of the present invention may be in the form of a tablet, (including a suspension tablet, a fast-melt tablet, a bite-disintegration tablet, a rapid-disintegration tablet, an effervescent tablet, or a caplet), a pill, a powder (including a sterile packaged powder, a dispensable powder, or an effervescent powder), a capsule (including both soft or hard capsules, e.g., capsules made from animal-derived gelatin or plant-derived HPMC, or “sprinkle capsules”), solid dispersion, solid solution, bioerodible dosage form, controlled release formulations, pulsatile release dosage forms, multiparticulate dosage forms, pellets, granules, or an aerosol. In other embodiments, the pharmaceutical formulation is in the form of a powder. In still other embodiments, the pharmaceutical formulation is in the form of a tablet, including but not limited to, a fast-melt tablet. Additionally, pharmaceutical formulations of the present invention may be administered as a single capsule or in multiple capsule dosage form. In some embodiments, the pharmaceutical formulation is administered in two, or three, or four, capsules or tablets.

In some embodiments, solid dosage forms, e.g., tablets, effervescent tablets, and capsules, are prepared by mixing the active agent(s) of the present invention particles with one or more pharmaceutical excipients to form a bulk blend composition. When referring to these bulk blend compositions as homogeneous, it is meant that the active agent(s) of the present invention particles are dispersed evenly throughout the composition so that the composition may be readily subdivided into equally effective unit dosage forms, such as tablets, pills, and capsules. The individual unit dosages may also comprise film coatings, which disintegrate upon oral ingestion or upon contact with diluents. These the active agent(s) of the present invention formulations can be manufactured by conventional pharmaceutical techniques.

Conventional pharmaceutical techniques for preparation of solid dosage forms include, e.g., one or a combination of methods: (1) dry mixing, (2) direct compression, (3) milling, (4) dry or non-aqueous granulation, (5) wet granulation, or (6) fusion. See, e.g., Lachman et al., Theory and Practice of Industrial Pharmacy (1986). Other methods include, e.g., spray drying, pan coating, melt granulation, granulation, fluidized bed spray drying or coating (e.g., wurster coating), tangential coating, top spraying, tableting, extruding and the like.

The pharmaceutical solid dosage forms described herein can comprise the active agent(s) of the present invention compositions described herein and one or more pharmaceutically acceptable additives such as a compatible carrier, binder, complexing agent, ionic dispersion modulator, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof. In still other aspects, using standard coating procedures, such as those described in Remington's Pharmaceutical Sciences, 20th Edition (2000), a film coating is provided around the active agent(s) of the present invention formulation. In one embodiment, some or all of the active agent(s) of the present invention particles are coated. In another embodiment, some or all of the active agent(s) of the present invention particles are microencapsulated. In yet another embodiment, some or all of the active agent(s) of the present invention is amorphous material coated and/or microencapsulated with inert excipients. In still another embodiment, the active agent(s) of the present invention particles not microencapsulated and are uncoated.

Suitable carriers for use in the solid dosage forms described herein include, but are not limited to, acacia, gelatin, colloidal silicon dioxide, calcium glycerophosphate, calcium lactate, maltodextrin, glycerin, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, hydroxypropylmethylcellulose, hydroxypropylmethylcellulose acetate stearate, sucrose, microcrystalline cellulose, lactose, mannitol and the like.

Suitable filling agents for use in the solid dosage forms described herein include, but are not limited to, lactose, calcium carbonate, calcium phosphate, dibasic calciumphosphate, calcium sulfate, microcrystalline cellulose (e.g., Avicel®, Avicel® PH101, Avicel® PH102, Avicel® PH105, etc.), cellulose powder, dextrose, dextrates, dextran, starches, pregelatinized starch, hydroxypropylmethylcellulose (HPMC), hydroxypropylmethylcellulose phthalate, hydroxypropylmethylcellulose acetate stearate (HPMCAS), sucrose, xylitol, lactitol, mannitol, sorbitol, sodium chloride, polyethylene glycol, and the like.

If needed, suitable disintegrants for use in the solid dosage forms described herein include, but are not limited to, natural starch such as corn starch or potato starch, a pregelatinized starch such as National 1551 or Amijel®, or a sodium starch glycolate such as Promogel® or Explotab®, a cellulose such as a wood product, microcrystalline cellulose, e.g., Avicel®, Avicel® PH101, Avicel® PH102, Avicel® PH105, Elcema® P100, Emcocel®, Vivacel®, Ming Tia®, and Solka-Floc®, Ac-Di-Sol, methylcellulose, croscarmellose, or a cross-linked cellulose, such as cross-linked sodium carboxymethylcellulose (Ac-Di-Sol®), cross-linked carboxymethylcellulose, or cross-linked croscarmellose, a cross-linked starch such as sodium starch glycolate, a cross-linked polymer such as crosspovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

Binders impart cohesiveness to solid oral dosage form formulations: for powder filled capsule formulation, they aid in plug formation that can be filled into soft or hard shell capsules and in tablet formulation, binders ensure that the tablet remains intact after compression and help assure blend uniformity prior to a compression or fill step. Materials suitable for use as binders in the solid dosage forms described herein include, but are not limited to, carboxymethylcellulose, methylcellulose (e.g., Methocel®), hydroxypropylmethylcellulose (e.g. Hypromellose USP Pharmacoat-603, hydroxypropylmethylcellulose acetate stearate (Aqoate HS-LF and HS), hydroxyethylcellulose, hydroxypropylcellulose (e.g., Klucel®), ethylcellulose (e.g., Ethocel®), and microcrystalline cellulose (e.g., Avicel®), microcrystalline dextrose, amylose, magnesium aluminum silicate, polysaccharide acids, bentonites, gelatin, polyvinylpyrrolidone/vinyl acetate copolymer, crosspovidone, povidone, starch, pregelatinized starch, tragacanth, dextrin, a sugar, such as sucrose (e.g., Dipac®), glucose, dextrose, molasses, mannitol, sorbitol, xylitol (e.g., Xylitab®), lactose, a natural or synthetic gum such as acacia, tragacanth, ghatti gum, mucilage of isapol husks, starch, polyvinylpyrrolidone (e.g., Povidone® CL, Kollidon® CL, Polyplasdone® XL-10, and Povidone® K-12), larch arabogalactan, Veegum®, polyethylene glycol, waxes, sodium alginate, and the like. In general, binder levels of 20-70% are used in powder-filled gelatin capsule formulations. Binder usage level in tablet formulations is a function of whether direct compression, wet granulation, roller compaction, or usage of other excipients such as fillers which itself can act as moderate binder are used. Formulators skilled in art can determine the binder level for the formulations, but binder usage level of up to 70% in tablet formulations is common.

Suitable lubricants or glidants for use in the solid dosage forms described herein include, but are not limited to, stearic acid, calcium hydroxide, talc, corn starch, sodium stearyl fumarate, alkali-metal and alkaline earth metal salts, such as aluminum, calcium, magnesium, zinc, stearic acid, sodium stearates, magnesium stearate, zinc stearate, waxes, Stearowet®, boric acid, sodium benzoate, sodium acetate, sodium chloride, leucine, a polyethylene glycol or a methoxypolyethylene glycol such as Carbowax™, PEG 4000, PEG 5000, PEG 6000, propylene glycol, sodium oleate, glyceryl behenate, glyceryl palmitostearate, glyceryl benzoate, magnesium or sodium lauryl sulfate, and the like.

Suitable diluents for use in the solid dosage forms described herein include, but are not limited to, sugars (including lactose, sucrose, and dextrose), polysaccharides (including dextrates and maltodextrin), polyols (including mannitol, xylitol, and sorbitol), cyclodextrins and the like.

Non water-soluble diluents are compounds typically used in the formulation of pharmaceuticals, such as calcium phosphate, calcium sulfate, starches, modified starches and microcrystalline cellulose, and microcellulose (e.g., having a density of about 0.45 g/cm3, e.g. Avicel, powdered cellulose), and talc.

Suitable wetting agents for use in the solid dosage forms described herein include, for example, oleic acid, glyceryl monostearate, sorbitan monooleate, sorbitan monolaurate, triethanolamine oleate, polyoxyethylene sorbitan monooleate, polyoxyethylene sorbitan monolaurate, quaternary ammonium compounds (e.g., Polyquat 10®), sodium oleate, sodium lauryl sulfate, magnesium stearate, sodium docusate, triacetin, vitamin E TPGS and the like. Wetting agents include surfactants.

Suitable surfactants for use in the solid dosage forms described herein include, for example, docusate and its pharmaceutically acceptable salts, sodium lauryl sulfate, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like.

Suitable suspending agents for use in the solid dosage forms described here include, but are not limited to, polyvinylpyrrolidone, e.g., polyvinylpyrrolidone K12, polyvinylpyrrolidone K17, polyvinylpyrrolidone K25, or polyvinylpyrrolidone K30, polyethylene glycol, e.g., the polyethylene glycol can have a molecular weight of about 300 to about 6000, or about 3350 to about 4000, or about 7000 to about 18000, vinyl pyrrolidone/vinyl acetate copolymer (S630), sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, polyethoxylated sorbitan monolaurate, polyethoxylated sorbitan monolaurate, povidone and the like.

Suitable antioxidants for use in the solid dosage forms described herein include, for example, e.g., butylated hydroxytoluene (BHT), butylhydroxyanisole (BHA), sodium ascorbate, Vitamin E TPGS, ascorbic acid, sorbic acid and tocopherol.

The above-listed additives should be taken as merely exemplary, and not limiting, of the types of additives that can be included in solid dosage forms of the present invention. The amounts of such additives can be readily determined by one skilled in the art, according to the particular properties desired.

Compressed tablets are solid dosage forms prepared by compacting the bulk blend the active agent(s) of the present invention formulations described above. In various embodiments, compressed tablets which are designed to dissolve in the mouth will comprise one or more flavoring agents. In other embodiments, the compressed tablets will comprise a film surrounding the final compressed tablet. In some embodiments, the film coating can provide a delayed release of the active agent(s) of the present invention formulation. In other embodiments, the film coating aids in patient compliance (e.g., Opadry® coatings or sugar coating). Film coatings comprising Opadry® typically range from about 1% to about 3% of the tablet weight. Film coatings for delayed release usually comprise 2-6% of a tablet weight or 7-15% of a spray-layered bead weight. In other embodiments, the compressed tablets comprise one or more excipients.

A capsule may be prepared, e.g., by placing the bulk blend the active agent(s) of the present invention formulation, described above, inside of a capsule. In some embodiments, the active agent(s) of the present invention formulations (non-aqueous suspensions and solutions) are placed in a soft gelatin capsule. In other embodiments, the active agent(s) of the present invention formulations are placed in standard gelatin capsules or non-gelatin capsules such as capsules comprising HPMC. In other embodiments, the active agent(s) of the present invention formulations are placed in a sprinkle capsule, wherein the capsule may be swallowed whole or the capsule may be opened and the contents sprinkled on food prior to eating. In some embodiments of the present invention, therapeutic dose is split into multiple (e.g., two, three, or four) capsules. In some embodiments, the entire dose of the active agent(s) of the present invention formulation is delivered in a capsule form. For example, the capsule may comprise between about 100 mg to about 600 mg of the active agent(s) of the present invention. In some embodiments, the capsule may comprise between about 100 to about 500 mg of the active agent(s) of the present invention. In other embodiments, capsule may comprise about 300 mg to about 400 mg of the active agent(s) of the present invention.

Another useful capsule has a shell comprising the material of the rate-limiting membrane, including any of the coating materials previously discussed, and filled with the active agent(s) of the present invention particles. A particular advantage of this configuration is that the capsule may be prepared independently of the active agent(s) of the present invention particles, thus process conditions that would adversely affect the drug can be used to prepare the capsule. A preferred embodiment is a capsule having a shell made of a porous or a pH-sensitive polymer made by a thermal forming process. An especially preferred embodiment is a capsule shell in the form of an asymmetric membrane; i.e., a membrane that has a thin skin on one surface and most of whose thickness is constituted of a highly permeable porous material. A preferred process for preparation of asymmetric membrane capsules comprises a solvent exchange phase inversion, wherein a solution of polymer, coated on a capsule-shaped mold, is induced to phase-separate by exchanging the solvent with a miscible non-solvent.

In yet another embodiment, spray layered active agent(s) of the present invention particles are filled in a capsule. An exemplary process for manufacturing the spray layered the active agent(s) of the present invention is the fluidized bed spraying process. The active agent(s) of the present invention suspensions or the active agent(s) of the present invention complex suspensions described above are sprayed onto sugar or microcrystalline cellulose (MCC) beads (20-35 mesh) with Wurster column insert at an inlet temperature of 50° C. to 60° C. and air temp of 30° C. to 50° C. A 15 to 20 wt % total solids content suspension containing 45 to 80 wt % the active agent(s) of the present invention, 10 to 25 wt % hydroxymethylpropylcellulose, 0.25 to 2 wt % of SLS, 10 to 18 wt % of sucrose, 0.01 to 0.3 wt % simethicone emulsion (30% emulsion) and 0.3 to 10% NaCl, based on the total weight of the solid content of the suspension, are sprayed (bottom spray) onto the beads through 1.2 mm nozzles at 10 mL/min and 1.5 bar of pressure until a layering of 400 to 700% wt % is achieved as compared to initial beads weight. The resulting spray layered the active agent(s) of the present invention particles or the active agent(s) of the present invention complex particles comprise about 30 to 70 wt % of the active agent(s) of the present invention based on the total weight of the particles. In one embodiment the capsule is a size 0 soft gelatin capsule In one embodiment, the capsule is a swelling plug device. In another embodiment, the swelling plug device is further coated with cellulose acetate phthalate or copolymers of methacrylic acid and methylmethacrylate.

In some embodiments the capsule includes at least 250 mg (or at least 300 mg or at least 400 mg) the active agent(s) of the present invention and has a total weight of less than 800 mg (or less that 700 mg). The capsule may contain a plurality of the active agent(s) of the present invention-containing beads, for example spray layered beads. In some embodiments the beads are 12-25% the active agent(s) of the present invention by weight. In some embodiments some or all of the active agent(s) of the present invention containing beads are coated with a coating comprising 6 to 15% (or 8 to 12%) of the total bead weight. Optimization work typically involves lower loading levels and the beads constitute 30 to 60% of the finished bead weight. The capsule may contain a granulated composition, wherein the granulated composition comprises the active agent(s) of the present invention.

The capsule may be pulsatile release the active agent(s) of the present invention oral dosage form, comprising: (a) a first dosage unit comprising a first the active agent(s) of the present invention dose that is released substantially immediately following oral administration of the dosage form to a patient; (b) a second dosage unit comprising a second the active agent(s) of the present invention dose that is released approximately 3 to 7 hours following administration of the dosage form to a patient. For pulsatile release capsules containing beads the beads can be coated with a coating comprising 6 to 15% (or 8 to 12%) of the total bead weight. In some embodiments the coating is a coating that is insoluble at pH 1 to 2 and soluble at pH greater than 5.5.

In certain embodiments, the formulation may comprise a pulsatile release capsule comprising at least two active agents. This pulsatile release capsule may contain a plurality of beads in which some beads are immediate release beads and other beads are formulated, for example with the use of a coating, for modified release, typically from about 3 to about 10 hours after administration. In other embodiments the pulsatile release capsule contains a plurality of beads formulated for modified release and the active agent(s) of the present invention powder, for example spray granulated the active agent(s) of the present invention, for immediate release.

In some embodiments, the release of the active agent(s) of the present invention particles can be modified with a modified release coating, such as an enteric coating using cellulose acetate phthalate or a sustained release coating comprising copolymers of methacrylic acid and methylmethacrylate. In one embodiment, the enteric coating may be present in an amount of about 0.5 to about 15 wt %, more specifically, about 8 to about 12 wt %, based on the weight of, e.g., the spray layered particles. In one embodiment, the spray layered particles coated with the delayed and/or sustained release coatings can be filled in a modified release capsule in which both enteric coated and immediate release the active agent(s) of the present invention beads are filled into a soft gelatin capsule. Additional suitable excipients may also be filled with the coated particles in the capsule. The uncoated particles release the active agent(s) of the present invention immediately upon administration while the coated particles do not release the active agent(s) of the present invention until these particles reach intestine. By controlling the ratios of the coated and uncoated particles, desirable pulsatile release profiles may be obtained. In some embodiments, the ratios between the uncoated and the coated particles are e.g., 20/80, or 30/70, or 40/60, or 50/50, w/w to obtain desirable release.

In some embodiments, the spray layered active agent(s) of the present invention particles can be compressed into tablets with commonly used pharmaceutical excipients. Any appropriate apparatus for forming the coating can be used to make the enteric coated tablets, e.g., fluidized bed coating using a Wurster column, powder layering in coating pans or rotary coaters; dry coating by double compression technique; tablet coating by film coating technique, and the like. See, e.g., U.S. Pat. No. 5,322,655; Remington's Pharmaceutical Sciences Handbook: Chapter 90 “Coating of Pharmaceutical Dosage Forms”, 1990.

In various embodiments, the spray layered the active agent(s) of the present invention described above and one or more excipients are dry blended and compressed into a mass, such as a tablet, having a hardness sufficient to provide a pharmaceutical composition that substantially disintegrates within less than about 30 minutes, less than about 35 minutes, less than about 40 minutes, less than about 45 minutes, less than about 50 minutes, less than about 55 minutes, or less than about 60 minutes, after oral administration, thereby releasing the active agent(s) of the present invention formulation into the gastrointestinal fluid.

In other embodiments, the spray layered the active agent(s) of the present invention particles or spray layered the active agent(s) of the present invention complex particles with enteric coatings described above and one or more excipients are dry blended and compressed into a mass, such as a tablet. In one embodiment, the enteric coated particles in the tablet substantially avoids release of the active agent(s) of the present invention, for example less than 15 wt %, in the stomach but releases substantially all the active agent(s) of the present invention (enterically or sustained release coated), for example, greater than 80 wt %, in the intestine.

In yet other embodiments, a pulsatile release the active agent(s) of the present invention formulation comprises a first dosage unit comprising a formulation made from the active agent(s) of the present invention containing granules made from a spray drying or spray granulated procedure or a formulation made from the active agent(s) of the present invention complex containing granules made from a spray drying or spray granulated procedure without enteric or sustained release coatings and a second dosage unit comprising spray layered the active agent(s) of the present invention particles or spray layered the active agent(s) of the present invention complex particles with enteric or sustained release coatings. In one embodiment, the first dosage unit and the second dosage unit are wet or dry blended and compressed into a mass to make a pulsatile release tablet.

In another embodiment, binding, lubricating and disintegrating agents are blended (wet or dry) to the spray layered the active agent(s) of the present invention to make a compressible blend. The first and the second dosage units are compressed separately and then compressed together to form a bilayer tablet. In yet another embodiment, the first dosage unit is in the form of an overcoat and completely covers the second dosage unit.

Roller compaction, which involves dry granulation of single powder or a blended mixture of powders by the use of pressure to form dense compacts (the compacts are subsequently milled to a desired particle size), provides another alternative. It is a simple process that is readily available for use, and does not involved the use of solvents for granulation. Thus, roller compaction eliminates the exposure of sensitive active pharmaceutical ingredients to moisture and drying. Roller compaction can also provide some enhanced stability and taste-masking characteristics to active pharmaceutical by diluting and isolating such components in a granulated matrix of compatible ingredients. Roller compaction also imparts increased density and flow to the powder.

Extrusion/spheronization is another method that involves wet massing of active pharmaceutical ingredients, followed by the extrusion of the wet mass through a perforated plate to produce short cylindrical rods. These rods are subsequently placed into a rapidly rotating spheronizer to shape the cylindrical rods into uniform spheres. The spheres are subsequently dried using a fluid bed drier and then coated with a functional coating using a fluid bed equipped with a Wurster insert and spray nozzle.

In other embodiments a powder comprising the active agent(s) of the present invention formulations described herein may be formulated to comprise one or more pharmaceutical excipients and flavors. Such a powder may be prepared, for example, by mixing the active agent(s) of the present invention formulation and optional pharmaceutical excipients to form a bulk blend composition. Additional embodiments also comprise a suspending agent and/or a wetting agent. This bulk blend is uniformly subdivided into unit dosage packaging or multi-dosage packaging units. The term “uniform” means the homogeneity of the bulk blend is substantially maintained during the packaging process

In still other embodiments, effervescent powders are also prepared in accordance with the present invention. Effervescent salts have been used to disperse medicines in water for oral administration. Effervescent salts are granules or coarse powders containing a medicinal agent in a dry mixture, usually composed of sodium bicarbonate, citric acid and/or tartaric acid. When salts of the present invention are added to water, the acids and the base react to liberate carbon dioxide gas, thereby causing “effervescence.” Examples of effervescent salts include, e.g: sodium bicarbonate or a mixture of sodium bicarbonate and sodium carbonate, citric acid and/or tartaric acid. Any acid-base combination that results in the liberation of carbon dioxide can be used in place of the combination of sodium bicarbonate and citric and tartaric acids, as long as the ingredients were suitable for pharmaceutical use and result in a pH of about 6.0 or higher.

In certain embodiments, ingredients (including or not including the active agent(s)) of the invention are wet granulated. The individual steps in the wet granulation process of tablet preparation include milling and sieving of the ingredients, dry powder mixing, wet massing, granulation, drying and final grinding. In various embodiments, the active agent(s) of the present invention composition is added to the other excipients of the pharmaceutical formulation after they have been wet granulated.

Alternatively, the ingredients may be subjected to dry granulation, e.g., via compressing a powder mixture into a rough tablet or “slug” on a heavy-duty rotary tablet press. The slugs are then broken up into granular particles by a grinding operation, usually by passage through an oscillation granulator. The individual steps include mixing of the powders, compressing (slugging) and grinding (slug reduction or granulation). No wet binder or moisture is involved in any of the steps. In some embodiments, the active agent(s) of the present invention formulation is dry granulated with other excipients in the pharmaceutical formulation. In other embodiments, the active agent(s) of the present invention formulation is added to other excipients of the pharmaceutical formulation after they have been dry granulated.

In other embodiments, the formulation of the present invention formulations described herein is a solid dispersions. Methods of producing such solid dispersions are known in the art and include, but are not limited to, for example, U.S. Pat. Nos. 4,343,789, 5,340,591, 5,456,923, 5,700,485, 5,723,269, and U.S. Pub. Appl 2004/0013734, each of which is specifically incorporated by reference. In some embodiments, the solid dispersions of the invention comprise both amorphous and non-amorphous the active agent(s) of the present invention and can have enhanced bioavailability as compared to conventional the active agent(s) of the present invention formulations. In still other embodiments, the active agent(s) of the present invention formulations described herein are solid solutions. Solid solutions incorporate a substance together with the active agent and other excipients such that heating the mixture results in dissolution of the drug and the resulting composition is then cooled to provide a solid blend which can be further formulated or directly added to a capsule or compressed into a tablet. Methods of producing such solid solutions are known in the art and include, but are not limited to, for example, U.S. Pat. Nos. 4,151,273, 5,281,420, and 6,083,518, each of which is specifically incorporated by reference.

The pharmaceutical solid oral dosage forms comprising the active agent(s) of the present invention described herein can be further formulated to provide a modified or controlled release of the active agent(s) of the present invention. In some embodiments, the solid dosage forms described herein can be formulated as a delay release dosage form such as and enteric coated delayed release oral dosage forms, i.e., as an oral dosage form of a pharmaceutical composition as described herein which utilizes an enteric coating to affect release in the small intestine of the gastrointestinal tract. The enteric coated dosage form may be a compressed or molded or extruded tablet/mold (coated or uncoated) containing granules, powder, pellets, beads or particles of the active ingredient and/or other composition components, which are themselves coated or uncoated. The enteric coated oral dosage form may also be a capsule (coated or uncoated) containing pellets, beads or granules of the solid carrier or the composition, which are themselves coated or uncoated. Enteric coatings may also be used to prepare other controlled release dosage forms including extended release and pulsatile release dosage forms.

In other embodiments, the active agent(s) of the formulations described herein are delivered using a pulsatile dosage form. Pulsatile dosage forms comprising the active agent(s) of the present invention formulations described herein may be administered using a variety of formulations known in the art. For example, such formulations include, but are not limited to, those described in U.S. Pat. Nos. 5,011,692, 5,017,381, 5,229,135, and 5,840,329, each of which is specifically incorporated by reference. Other dosage forms suitable for use with the active agent(s) of the present invention formulations are described in, for example, U.S. Pat. Nos. 4,871,549, 5,260,068, 5,260,069, 5,508,040, 5,567,441 and 5,837,284, all of which are specifically incorporated by reference. In one embodiment, the controlled release dosage form is pulsatile release solid oral dosage form comprising at least two groups of particles, each containing active agent(s) of the present invention as described herein. The first group of particles provides a substantially immediate dose of the active agent(s) of the present invention upon ingestion by a subject. The first group of particles can be either uncoated or comprise a coating and/or sealant. The second group of particles comprises coated particles, which comprise from about 2% to about 75%, preferably from about 2.5% to about 70%, and more preferably from about 40% to about 70%, by weight of the total dose of the active agent(s) of the present invention in said formulation, in admixture with one or more binders. The coating comprises a pharmaceutically acceptable ingredient in an amount sufficient to provide a delay of from about 2 hours to about 7 hours following ingestion before release of the second dose. Suitable coatings include one or more differentially degradable coatings such as, by way of example only, pH sensitive coatings (enteric coatings) such as acrylic resins (e.g., Eudragit® EPO, Eudragit® L30D-55, Eudragit® FS 30D Eudragit® L100-55, Eudragit® L100, Eudragit® S100, Eudragit® RD100, Eudragit® E100, Eudragit® L12.5, Eudragit® S12.5, and Eudragit® NE30D, Eudragit® NE 40D®) either alone or blended with cellulose derivatives, e.g., ethylcellulose, or non-enteric coatings having variable thickness to provide differential release of the active agent(s) of the present invention formulation.

Many other types of controlled release systems known to those of ordinary skill in the art and are suitable for use with the active agent(s) of the present invention formulations described herein. Examples of such delivery systems include, e.g., polymer-based systems, such as polylactic and polyglycolic acid, polyanhydrides and polycaprolactone, cellulose derivatives (e.g., ethyl cellulose), porous matrices, nonpolymer-based systems that are lipids, including sterols, such as cholesterol, cholesterol esters and fatty acids, or neutral fats, such as mono-, di- and triglycerides; hydrogel release systems; silastic systems; peptide-based systems; wax coatings, bioerodible dosage forms, compressed tablets using conventional binders and the like. See, e.g., Liberman et al., Pharmaceutical Dosage Forms, 2 Ed., Vol. 1, pp. 209-214 (1990); Singh et al., Encyclopedia of Pharmaceutical Technology, 2nd Ed., pp. 751-753 (2002); U.S. Pat. Nos. 4,327,725, 4,624,848, 4,968,509, 5,461,140, 5,456,923, 5,516,527, 5,622,721, 5,686,105, 5,700,410, 5,977,175, 6,465,014 and 6,932,983, each of which is specifically incorporated by reference. In some embodiments, materials include shellac, acrylic polymers, cellulosic derivatives, polyvinyl acetate phthalate, and mixtures thereof. In other embodiments, materials include Eudragit® series E, L, RL, RS, NE, L, L300, S, 100-55, cellulose acetate phthalate, Aquateric, cellulose acetate trimellitate, ethyl cellulose, hydroxypropylmethylcellulose phthalate, hydroxypropylmethylcellulose acetate succinate, polyvinyl acetate phthalate, and Cotteric.

The controlled release systems may utilize a hydrophilic polymer, including but not limited to a water swellable polymer (e.g., a natural or synthetic gum). The hydrophilic polymer may be any pharmaceutically acceptable polymer which swells and expands in the presence of water to slowly release the active agent(s) of the present invention. These polymers include polyethylene oxide, methylcellulose, hydroxypropylcellulose, hydroxypropylmethylcellulose and the like. In a preferred embodiment, the water swellable polymer will be polyethylene oxide (obtained from Union Carbide Corporation under the trade name Polyox WSR Coagulant or Polyox WSR N 80). These materials form a viscous gel in water or other solvent system at a sufficient concentration to control the release of the active agent(s) of the present invention. This will generally require a concentration of the pharmaceutically acceptable, water swellable polymer of about 0-50% by weight of the compressed, uncoated tablet.

The performance of acrylic polymers (primarily their solubility in biological fluids) can vary based on the degree and type of substitution. Examples of suitable acrylic polymers include methacrylic acid copolymers and ammonia methacrylate copolymers. The Eudragit series E, L, S, RL, RS and NE (Rohm Pharma) are available as solubilized in organic solvent, aqueous dispersion, or dry powders. The Eudragit series RL, NE, and RS are insoluble in the gastrointestinal tract but are permeable and are used primarily for colonic targeting. The Eudragit series E dissolve in the stomach. The Eudragit series L, L-30D and S are insoluble in stomach and dissolve in the intestine; Opadry Enteric are also insoluble in stomach and dissolve in the intestine.

Examples of suitable cellulose derivatives are: ethyl cellulose; reaction mixtures of partial acetate esters of cellulose with phthalic anhydride. The performance can vary based on the degree and type of substitution. Cellulose acetate phthalate (CAP) dissolves in pH >6. Aquateric (FMC) is an aqueous based system and is a spray dried CAP psuedolatex with particles <1 μm. Other components in Aquateric can include pluronics, Tweens, and acetylated monoglycerides. Other suitable cellulose derivatives include: cellulose acetate trimellitate (Eastman); methylcellulose (Pharmacoat, Methocel); hydroxypropylmethyl cellulose phthalate (HPMCP); hydroxypropylmethyl cellulose succinate (HPMCS); and hydroxypropylmethylcellulose acetate succinate (e.g., AQOAT (Shin Etsu)). The performance can vary based on the degree and type of substitution. For example, HPMCP such as, HP-50, HP-55, HP-55S, HP-55F grades are suitable. The performance can vary based on the degree and type of substitution. For example, suitable grades of hydroxypropylmethylcellulose acetate succinate include, but are not limited to, AS-LG (LF), which dissolves at pH 5, AS-MG (MF), which dissolves at pH 5.5, and AS-HG (HF), which dissolves at higher pH. These polymers are offered as granules, or as fine powders for aqueous dispersions.

In some embodiments, the coating can, and usually does, contain a plasticizer and possibly other coating excipients such as colorants, talc, and/or magnesium stearate, which are well known in the art. Suitable plasticizers include triethyl citrate (Citroflex 2), triacetin (glyceryl triacetate), acetyl triethyl citrate (Citroflec A2), Carbowax 400 (polyethylene glycol 400), diethyl phthalate, tributyl citrate, acetylated monoglycerides, glycerol, fatty acid esters, propylene glycol, and dibutyl phthalate. In particular, anionic carboxylic acrylic polymers usually will contain 10-25% by weight of a plasticizer, especially dibutyl phthalate, polyethylene glycol, triethyl citrate and triacetin. Conventional coating techniques such as spray or pan coating are employed to apply coatings. The coating thickness must be sufficient to ensure that the oral dosage form remains intact until the desired site of topical delivery in the intestinal tract is reached.

In some embodiments, formulations are provided comprising the active agent(s) of the present invention particles described herein and at least one dispersing agent or suspending agent for oral administration to a subject. The formulation may be a powder and/or granules for suspension, and upon admixture with water, a substantially uniform suspension is obtained. As described herein, the aqueous dispersion can comprise amorphous and non-amorphous the active agent(s) of the present invention particles of consisting of multiple effective particle sizes such that the active agent(s) of the present invention particles having a smaller effective particle size are absorbed more quickly and the active agent(s) of the present invention particles having a larger effective particle size are absorbed more slowly. In certain embodiments the aqueous dispersion or suspension is an immediate release formulation. In another embodiment, an aqueous dispersion comprising amorphous the active agent(s) of the present invention particles is formulated such that a portion of the active agent(s) of the present invention particles are absorbed within, e.g., about 3 hours after administration and about 90% of the active agent(s) of the present invention particles are absorbed within, e.g., about 10 hours after administration. In other embodiments, addition of a complexing agent to the aqueous dispersion results in a larger span of the active agent(s) of the present invention containing particles to extend the drug absorption phase such that 50-80% of the particles are absorbed in the first 3 hours and about 90% are absorbed by about 10 hours. Dosage forms for oral administration can be aqueous suspensions selected from the group including, but not limited to, pharmaceutically acceptable aqueous oral dispersions, emulsions, solutions, and syrups. See, e.g., Singh et al., Encyclopedia of Pharmaceutical Technology, 2nd Ed., pp. 754-757 (2002). In addition to the active agent(s) of the present invention particles, the liquid dosage forms may comprise additives, such as: (a) disintegrating agents; (b) dispersing agents; (c) wetting agents; (d) at least one preservative, (e) viscosity enhancing agents, (f) at least one sweetening agent, and (g) at least one flavoring agent.

Examples of disintegrating agents for use in the aqueous suspensions and dispersions include, but are not limited to, a starch, e.g., a natural starch such as corn starch or potato starch, a pregelatinized starch such as National 1551 or Amijel®, or sodium starch glycolate such as Promogel® or Explotab®; a cellulose such as a wood product, microcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose; a cross-linked starch such as sodium starch glycolate; a cross-linked polymer such as crosspovidone; a cross-linked polyvinylpyrrolidone; alginate such as alginic acid or a salt of alginic acid such as sodium alginate; a clay such as Veegum® HV (magnesium aluminum silicate); a gum such as agar, guar, locust bean, Karaya, pectin, or tragacanth; sodium starch glycolate; bentonite; a natural sponge; a surfactant; a resin such as a cation-exchange resin; citrus pulp; sodium lauryl sulfate; sodium lauryl sulfate in combination starch; and the like.

In some embodiments, the dispersing agents suitable for the aqueous suspensions and dispersions described herein are known in the art and include, for example, hydrophilic polymers, electrolytes, Tween® 60 or 80, PEG, polyvinylpyrrolidone (PVP; commercially known as Plasdone®), and the carbohydrate-based dispersing agents such as, for example, hydroxypropylcellulose and hydroxypropylcellulose ethers (e.g., HPC, HPC-SL, and HPC-L), hydroxypropylmethylcellulose and hydroxypropylmethylcellulose ethers (e.g. HPMC K100, HPMC K4M, HPMC K15M, and HPMC K100M), carboxymethylcellulose sodium, methylcellulose, hydroxyethylcellulose, hydroxypropylmethylcellulose phthalate, hydroxypropylmethylcellulose acetate stearate, noncrystalline cellulose, magnesium aluminum silicate, triethanolamine, polyvinyl alcohol (PVA), polyvinylpyrrolidone/vinyl acetate copolymer (Plasdone®, e.g., S-630), 4-(1,1,3,3-tetramethylbutyl)-phenol polymer with ethylene oxide and formaldehyde (also known as tyloxapol), poloxamers (e.g., Pluronics F68®, F88®, and F108®, which are block copolymers of ethylene oxide and propylene oxide); and poloxamines (e.g., Tetronic 908®, also known as Poloxamine 908®, which is a tetrafunctional block copolymer derived from sequential addition of propylene oxide and ethylene oxide to ethylenediamine (BASF Corporation, Parsippany, N.J.)). In other embodiments, the dispersing agent is selected from a group not comprising one of the following agents: hydrophilic polymers; electrolytes; Tween® 60 or 80; PEG; polyvinylpyrrolidone (PVP); hydroxypropylcellulose and hydroxypropyl cellulose ethers (e.g., HPC, HPC-SL, and HPC-L); hydroxypropyl methylcellulose and hydroxypropyl methylcellulose ethers (e.g. HPMC K100, HPMC K4M, HPMC K15M, HPMC K100M, and Pharmacoat® USP 2910 (Shin-Etsu)); carboxymethylcellulose sodium; methylcellulose; hydroxyethylcellulose; hydroxypropylmethyl-cellulose phthalate; hydroxypropylmethyl-cellulose acetate stearate; non-crystalline cellulose; magnesium aluminum silicate; triethanolamine; polyvinyl alcohol (PVA); 4-(1,1,3,3-tetramethylbutyl)-phenol polymer with ethylene oxide and formaldehyde; poloxamers (e.g., Pluronics F68®, F88®, and F108®, which are block copolymers of ethylene oxide and propylene oxide); or poloxamines (e.g., Tetronic 908®, also known as Poloxamine 908®).

Wetting agents (including surfactants) suitable for the aqueous suspensions and dispersions described herein are known in the art and include, but are not limited to, acetyl alcohol, glycerol monostearate, polyoxyethylene sorbitan fatty acid esters (e.g., the commercially available Tweens® such as e.g., Tween 20® and Tween 80® (ICI Specialty Chemicals)), and polyethylene glycols (e.g., Carbowaxs 3350® and 1450®, and Carpool 934® (Union Carbide)), oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, sodium lauryl sulfate, sodium docusate, triacetin, vitamin E TPGS, sodium taurocholate, simethicone, phosphotidylcholine and the like.

Suitable preservatives for the aqueous suspensions or dispersions described herein include, for example, potassium sorbate, parabens (e.g., methylparaben and propylparaben) and their salts, benzoic acid and its salts, other esters of parahydroxybenzoic acid such as butylparaben, alcohols such as ethyl alcohol or benzyl alcohol, phenolic compounds such as phenol, or quaternary compounds such as benzalkonium chloride. Preservatives, as used herein, are incorporated into the dosage form at a concentration sufficient to inhibit microbial growth. In one embodiment, the aqueous liquid dispersion can comprise methylparaben and propylparaben in a concentration ranging from about 0.01% to about 0.3% methylparaben by weight to the weight of the aqueous dispersion and about 0.005% to about 0.03% propylparaben by weight to the total aqueous dispersion weight. In yet another embodiment, the aqueous liquid dispersion can comprise methylparaben from about 0.05 to about 0.1 weight % and propylparaben from about 0.01 to about 0.02 weight % of the aqueous dispersion.

Suitable viscosity enhancing agents for the aqueous suspensions or dispersions described herein include, but are not limited to, methyl cellulose, xanthan gum, carboxymethylcellulose, hydroxypropyl cellulose, hydroxypropylmethyl cellulose, Plasdone® S-630, carbomer, polyvinyl alcohol, alginates, acacia, chitosans and combinations thereof. The concentration of the viscosity enhancing agent will depend upon the agent selected and the viscosity desired.

Examples of natural and artificial sweetening agents suitable for the aqueous suspensions or dispersions described herein include, for example, acacia syrup, acesulfame K, alitame, anise, apple, aspartame, banana, Bavarian cream, berry, black currant, butterscotch, calcium citrate, camphor, caramel, cherry, cherry cream, chocolate, cinnamon, bubble gum, citrus, citrus punch, citrus cream, cotton candy, cocoa, cola, cool cherry, cool citrus, cyclamate, cylamate, dextrose, eucalyptus, eugenol, fructose, fruit punch, ginger, glycyrrhetinate, glycyrrhiza (licorice) syrup, grape, grapefruit, honey, isomalt, lemon, lime, lemon cream, monoammonium glyrrhizinate (MagnaSweet®), maltol, mannitol, maple, marshmallow, menthol, mint cream, mixed berry, neohesperidine DC, neotame, orange, pear, peach, peppermint, peppermint cream, Prosweet® Powder, raspberry, root beer, rum, saccharin, safrole, sorbitol, spearmint, spearmint cream, strawberry, strawberry cream, stevia, sucralose, sucrose, sodium saccharin, saccharin, aspartame, acesulfame potassium, mannitol, talin, sucralose, sorbitol, Swiss cream, tagatose, tangerine, thaumatin, tutti fruitti, vanilla, walnut, watermelon, wild cherry, wintergreen, xylitol, or any combination of these flavoring ingredients, e.g., anise-menthol, cherry-anise, cinnamon-orange, cherry-cinnamon, chocolate-mint, honey-lemon, lemon-lime, lemon-mint, menthol-eucalyptus, orange-cream, vanilla-mint, and mixtures thereof. In one embodiment, the aqueous liquid dispersion can comprise a sweetening agent or flavoring agent in a concentration ranging from about 0.0001% to about 10.0% the weight of the aqueous dispersion. In another embodiment, the aqueous liquid dispersion can comprise a sweetening agent or flavoring agent in a concentration ranging from about 0.0005% to about 5.0% wt % of the aqueous dispersion. In yet another embodiment, the aqueous liquid dispersion can comprise a sweetening agent or flavoring agent in a concentration ranging from about 0.0001% to 0.1 wt %, from about 0.001% to about 0.01 weight %, or from 0.0005% to 0.004% of the aqueous dispersion.

In addition to the additives listed above, the liquid the active agent(s) of the present invention formulations can also comprise inert diluents commonly used in the art, such as water or other solvents, solubilizing agents, and emulsifiers.

In some embodiments, formulations described herein can be self-emulsifying drug delivery systems (SEDDS). Emulsions are dispersions of one immiscible phase in another, usually in the form of droplets. Generally, emulsions are created by vigorous mechanical dispersion. SEDDS, as opposed to emulsions or microemulsions, spontaneously form emulsions when added to an excess of water without any external mechanical dispersion or agitation. An advantage of SEDDS is that only gentle mixing is required to distribute the droplets throughout the solution. Additionally, water or the aqueous phase can be added just prior to administration, which ensures stability of an unstable or hydrophobic active ingredient. Thus, the SEDDS provides an effective delivery system for oral and parenteral delivery of hydrophobic active ingredients. SEDDS may provide improvements in the bioavailability of hydrophobic active ingredients. Methods of producing self-emulsifying dosage forms are known in the art include, but are not limited to, for example, U.S. Pat. Nos. 5,858,401, 6,667,048, and 6,960,563, each of which is specifically incorporated by reference. Exemplary emulsifiers are ethyl alcohol, isopropyl alcohol, ethyl carbonate, ethyl acetate, benzyl alcohol, benzyl benzoate, propylene glycol, 1,3-butyleneglycol, dimethylformamide, sodium lauryl sulfate, sodium doccusate, cholesterol, cholesterol esters, taurocholic acid, phosphotidylcholine, oils, such as cottonseed oil, groundnut oil, corn germ oil, olive oil, castor oil, and sesame oil, glycerol, tetrahydrofurfuryl alcohol, polyethylene glycols, fatty acid esters of sorbitan, or mixtures of these substances, and the like.

In certain embodiments, the active agent(s) may be administered via Intranasal formulations which are known in the art and are described in, for example, U.S. Pat. Nos. 4,476,116, 5,116,817 and 6,391,452, each of which is specifically incorporated by reference. The active agent(s) of the present invention prepared according to these and other techniques well-known in the art are prepared as solutions in saline, employing benzyl alcohol or other suitable preservatives, fluorocarbons, and/or other solubilizing or dispersing agents known in the art. See, for example, Ansel, H. C. et al., Pharmaceutical Dosage Forms and Drug Delivery Systems, Sixth Ed. (1995). Preferably these compositions and formulations are prepared with suitable nontoxic pharmaceutically acceptable ingredients. These ingredients are known to those skilled in the preparation of nasal dosage forms and some of these can be found in REMINGTON: THE SCIENCE AND PRACTICE OF PHARMACY, 21st edition, 2005, a standard reference in the field. The choice of suitable carriers is highly dependent upon the exact nature of the nasal dosage form desired, e.g., solutions, suspensions, ointments, or gels. Nasal dosage forms generally contain large amounts of water in addition to the active ingredient. Minor amounts of other ingredients such as pH adjusters, emulsifiers or dispersing agents, preservatives, surfactants, gelling agents, complexing agents or buffering and other stabilizing and solubilizing agents may also be present. Preferably, the nasal dosage form should be isotonic with nasal secretions.

In certain embodiments, the active agent(s) may be administered via buccal formulations such as, but are not limited to, U.S. Pat. Nos. 4,229,447, 4,596,795, 4,755,386, and 5,739,136, each of which is specifically incorporated by reference. In addition, the buccal dosage forms described herein can further comprise a bioerodible (hydrolyzable) polymeric carrier that may also serves to adhere the dosage form to the buccal mucosa. The buccal dosage form is fabricated so as to erode gradually over a predetermined time period, wherein the active agent(s) of the present invention delivery is provided essentially throughout. Buccal drug delivery, as will be appreciated by those skilled in the art, avoids the disadvantages encountered with oral drug administration, e.g., slow drug absorption, degradation of the active agent by fluids present in the gastrointestinal tract and/or first-pass inactivation in the liver. With regard to the bioerodible (hydrolysable) polymeric carrier, it will be appreciated that virtually any such carrier can be used, so long as the desired drug release profile is not comprised, and the carrier is compatible with the active agent(s) of the present invention and any other components that may be present in the buccal dosage unit. Generally, the polymeric carrier comprises hydrophilic (water-soluble and water-swellable) polymers that adhere to the wet surface of the buccal mucosa. Examples of polymeric carriers useful herein include acrylic acid polymers and co, e.g., those known as “carbomers” (Carbopol®, which may be obtained from B.F. Goodrich, is one such polymer). Other components may also be incorporated into the buccal dosage forms described herein include, but are not limited to, disintegrants, diluents, binders, lubricants, flavoring, colorants, preservatives, and the like.

In certain embodiments, the active agent(s) may be administered via transdermal formulations using a variety of devices which have been described in the art. For example, such devices include, but are not limited to, U.S. Pat. Nos. 3,598,122, 3,598,123, 3,710,795, 3,731,683, 3,742,951, 3,814,097, 3,921,636, 3,972,995, 3,993,072, 3,993,073, 3,996,934, 4,031,894, 4,060,084, 4,069,307, 4,077,407, 4,201,211, 4,230,105, 4,292,299, 4,292,303, 5,336,168, 5,665,378, 5,837,280, 5,869,090, 6,923,983, 6,929,801 and 6,946,144, each of which is specifically incorporated by reference in its entirety. In some embodiments, the transdermal delivery device used with the active agent(s) of the present invention described herein can comprise a power source, radio frequency, or a brief electrical current to micro-electrodes in the skin creating “channels” or “pores” in the stratum corneum to facilitate the delivery of the active agent(s) of the present invention formulation, such methods are known in the art and are described in, for example U.S. Pat. Nos. 6,611,706, 6,708,060, and 6,711,435, each of which is specifically incorporated by reference in its entirety. In other embodiments, the transdermal delivery device can comprise a means for porating the stratum corneum, e.g., micro-lancing, application of sonic energy, or hydraulic puncturing, to facilitate the delivery of the active agent(s) of the present invention, such methods are known in the art and are described in, for example, U.S. Pat. Nos. 6,142,939 and 6,527,716, each of which is specifically incorporated by reference in its entirety. The pores described by the methods herein are typically about 20-50 microns in depth and to not extend into areas of innervation or vascularization.

The transdermal dosage forms described herein may incorporate certain pharmaceutically acceptable excipients which are conventional in the art. In general, the transdermal formulations described herein comprise at least three components: (1) the active agent(s) of the present invention; (2) a penetration enhancer; and (3) an aqueous adjuvant. In addition, transdermal formulations can include additional components such as, but not limited to, gelling agents, creams and ointment bases, and the like. In some embodiments, the transdermal formulation can further comprise a woven or non-woven backing material to enhance drug absorption and prevent the removal of the transdermal formulation from the skin. In other embodiments, the transdermal formulations described herein can maintain a saturated or supersaturated state to promote diffusion into the skin.

The active agent(s) of the present invention formulations suitable for intramuscular, subcutaneous, or intravenous injection may comprise physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including water, ethanol, polyols (propylene glycol, polyethylene-glycol, glycerol, cremophor and the like), suitable mixtures thereof, vegetable oils (such as olive oil) and injectable organic esters such as ethyl oleate. Additionally, the active agent(s) of the present invention can be dissolved at concentrations of >1 mg/ml using water soluble beta cyclodextrins (e.g. beta-sulfobutyl-cyclodextrin and 2-hydroxypropylbetacyclodextrin. Proper fluidity can be maintained, for example, by the use of a coating such as lecithin, by the maintenance of the required particle size in the case of dispersions, and by the use of surfactants. The active agent(s) of the present invention formulations suitable for subcutaneous injection may also contain additives such as preserving, wetting, emulsifying, and dispensing agents. Prevention of the growth of microorganisms can be ensured by various antibacterial and antifungal agents, such as parabens, benzoic acid, benzyl alcohol, chlorobutanol, phenol, sorbic acid, and the like. It may also be desirable to include isotonic agents, such as sugars, sodium chloride, and the like. Prolonged drug absorption of the injectable pharmaceutical form can be brought about by the use of agents delaying absorption, such as aluminum monostearate and gelatin. The active agent(s) of the present invention suspension formulations designed for extended release via subcutaneous or intramuscular injection can avoid first pass metabolism and lower dosages of the active agent(s) of the present invention will be necessary to maintain plasma levels of about 50 ng/ml. In such formulations, the particle size of the active agent(s) of the present invention particles and the range of the particle sizes of the active agent(s) of the present invention particles can be used to control the release of the drug by controlling the rate of dissolution in fat or muscle.

The particular choice of active agents used will depend upon the diagnosis of the attending physicians and their judgment of the condition of the patient and the appropriate treatment protocol. Each of the active agents may be administered concurrently (e.g., simultaneously, essentially simultaneously or within the same treatment protocol) or sequentially, depending upon the nature of the proliferative disease, the condition of the patient, and the actual choice of compounds used. The determination of the order of administration, and the number of repetitions of administration of each active agent during a treatment protocol, is well within the knowledge of the skilled physician after evaluation of the disease being treated and the condition of the patient.

It is understood that the dosage regimen to treat, prevent, or ameliorate the condition(s) for which relief is sought, can be modified in accordance with a variety of factors. These factors include the disorder from which the subject suffers, as well as the age, weight, sex, diet, and medical condition of the subject. Thus, the dosage regimen actually employed can vary widely and therefore can deviate from the dosage regimens set forth herein.

The pharmaceutical agents which make up the combination therapy disclosed herein may be a combined dosage form or in separate dosage forms intended for substantially simultaneous administration. The pharmaceutical agents that make up the combination therapy may also be administered sequentially, with either therapeutic compound being administered by a regimen calling for two-step administration. The two-step administration regimen may call for sequential administration of the active agents or spaced-apart administration of the separate active agents. The time period between the multiple administration steps may range from, a few minutes to several hours, depending upon the properties of each pharmaceutical agent, such as potency, solubility, bioavailability, plasma half-life and kinetic profile of the pharmaceutical agent. Circadian variation of the target molecule concentration may also determine the optimal dose interval.

### Detailed Description of the Preferred Embodiments

A unique combination of entry criteria for metabolic syndrome patients based on the guidelines recommended by the American Association of Clinical Endocrinologists will be used to select patients which would include moderate risk (MR) as well as high risk (HR) which would include: A1c> or =6.5, BMI> or =27 Kg/m2 (MR), BMI>=40 Kg/m2 (HR), Fasting plasma glucose (FPG)> or =126 mg/dL, 2 hour GTT (Glucose tolerance test)> or =200 mg/dL, non-fasting laboratory glucose> or =200 mg/dL, LDL-C> or =100 mg/dL, TG> or =150 mg/dL, non HDL-C> or =130 mg/dL, HDL-C< or =40 mg/dL, TC/HDL-C> or =3.5, ApoB> or =90 mg/dL, B.P. (blood pressure)> or =130/80 mm Hg. Other symptoms such as polydipsia, polyurea and/or polyphagia will also be considered.

In addition to the entry criteria, a set of risk stratification criteria will be used which would include or exclude patients which may contain: lack of adequate therapeutic and overall clinical benefit from previously prescribed mono- and combination therapies, BMI (BMI> or =27 Kg/m2), Age (> or =45 yrs), elevated B.P. (> or =130/80 mm Hg), abdominal adiposity (prototypical and/or pear-shaped), cigarette smoking, hsCRP> or =2.0, excessive alcohol intake, family history of type 2 diabetes, family history of coronary heart disease (CHD), pancreatis or hepatitis, chronic kidney disease or pre-existing renal impairment (Creatinine level> or =1.5 mg/dL in males and > or =1.4 mg/dL in females; eGFR (estimated glomerular filtration rate)< or =60 ml/min/1.73 m2), recent change in body weight (> or =5%), abnormal liver test (ALT> or =40), previous use of ACE inhibitors or ARBs etc.

All drug combinations will be titrated individually and in combinations for efficacy and safety in appropriate Type II diabetes and metabolic syndrome pre-clinical in vivo models. Final doses of drugs, FDC combinations and number of administrations daily depends on the patient's response and tolerance. The optimal dosage, FDC combination of drugs and modified release kinetics will be determined based on the overall desired clinical effect with minimal adverse side effects. Doses will be reduced or adjusted for patients' risk stratification criteria e.g. renal impairment.

The following examples illustrate various aspects of the present invention. They are not to be construed to limit the claims in any manner whatsoever.

### Example 1

### Anti-Inflammatory-Centric FDC Formulations (Type II Diabetes Drugs): Two drug combinations (IR & IR)

200 mg Ibuprofen or 250 mg Naproxen-Sodium or 325 mg of Aspirin or Aspirin-like drugs (e.g. Ascriptin, Ecotrin) or 50 mg to 400 mg Celecoxib or 200 mg Sulindac or an appropriate dose of other known anti-inflammatory drugs will be formulated in IR form in combination with anti-type II diabetes drugs such as Sulfonyl ureas such as 125 mg to 500 mg Chloropropamide or 125 mg to 500 mg Tolbutamide or 50 to 250 mg Tolazamide or 1.25 mg to 0.5 mg to 15 mg Glipizide or 0.5 mg to 20 mg Glyburide or 0.5 mg to 8 mg Glimepiride; or Biguanides such as 250 mg to 2000 mg Metformin; or Thiozolidinediones (TZDs) such as 7.5 mg to 30 mg Pioglitazone or 2 mg to 8 mg Rosiglitazone; or alpha glucosidase inhibitors such as 25 mg to 100 mg Acarbose or 12.5 mg 100 mg Miglitol; or DPPIV inhibitors such as 12.5 mg to 100 mg Sitagliptin, 3 mg to 25 mg Alogliptin or 2.5 mg Linagliptin; or SGLT-2 (Sodium Glucose co-transporters) inhibitors such as 2.5 mg to 10 mg Dapagliflozin or 50 to 300 mg Canagliflozin formulated in an IR form.

### Example 2

### Anti-Inflammatory-Centric FDC Formulations (Type II Diabetes Drugs): Two drug combinations (IR & ER)

200 mg Ibuprofen or 250 mg Naproxen-Sodium or 325 mg of Aspirin or Aspirin-like drugs (e.g. Ascriptin, Ecotrin) or 50 mg to 400 mg Celecoxib or 200 mg Sulindac or an appropriate dose of other known anti-inflammatory drugs will be formulated in IR form in combination with anti-type II diabetes drugs such as Sulfonyl ureas such as 500 mg Chloropropamide or 500 mg Tolbutamide or 100 mg Tolazamide or 20 mg Glipizide or 10 mg Glyburide or 8 mg Glimepiride; or Biguanides such as 500 mg Metformin; or Thiozolidinediones (TZDs) such as 30 mg Pioglitazone or 8 mg Rosiglitazone; or alpha glucosidase inhibitors such as 100 mg Acarbose or 100 mg Miglitol; or DPP-IV inhibitors such as 250 mg Sitagliptin, 25 mg Alogliptin or 10 mg Linagliptin; or SGLT-2 (Sodium Glucose co-transporters) inhibitors such as 25 mg Dapagliflozin or 300 mg Canagliflozin in an ER form.

### Example 2

### Anti-Inflammatory-Centric FDC Formulations (Type II Diabetes Drugs): Two drug combinations (IR & DR)

200 mg Ibuprofen or 250 mg Naproxen-Sodium or 325 mg of Aspirin or Aspirin-like drugs (e.g. Ascriptin, Ecotrin) or 50 mg to 400 mg Celecoxib or 200 mg Sulindac or an appropriate dose of other known anti-inflammatory drugs will be formulated in IR form in combination with anti-type II diabetes drugs such as Sulfonyl ureas such as 125 mg to 500 mg Chloropropamide or 125 mg to 500 mg Tolbutamide or 50 to 250 mg Tolazamide or 1.25 mg to 0.5 mg to 15 mg Glipizide or 0.5 mg to 20 mg Glyburide or 0.5 mg to 8 mg Glimepiride; or Biguanides such as 250 mg to 2000 mg Metformin; or Thiozolidinediones (TZDs) such as 7.5 mg to 30 mg Pioglitazone or 2 mg to 8 mg Rosiglitazone; or alpha glucosidase inhibitors such as 25 mg to 100 mg Acarbose or 12.5 mg 100 mg Miglitol; or DPPIV inhibitors such as 12.5 mg to 100 mg Sitagliptin, 3 mg to 25 mg Alogliptin or 2.5 mg Linagliptin; or SGLT-2 (Sodium Glucose co-transporters) inhibitors such as 2.5 mg to 10 mg Dapagliflozin or 50 to 300 mg Canagliflozin formulated in an IR form.

### Example 3

### Three Drug Combinations (IR/IR/ER): Anti-Inflammatory/Sulfonyl Urea/Biguanides

200 mg Ibuprofen or 250 mg Naproxen-Sodium or 325 mg of Aspirin or Aspirin-like drugs (e.g. Ascriptin, Ecotrin) or 50 mg to 400 mg Celecoxib or 200 mg Sulindac or an appropriate dose of other known anti-inflammatory drugs will be formulated in IR form in combination with anti-type II diabetes drugs such as Sulfonyl ureas such as 125 mg to 500 mg Chloropropamide or 125 mg to 500 mg Tolbutamide or 50 to 250 mg Tolazamide or 1.25 mg to 0.5 mg to 15 mg Glipizide or 0.5 mg to 20 mg Glyburide or 0.5 mg to 8 mg Glimepiride in an IR form and Biguanides such as 250 mg to 2000 mg Metformin in an ER form.

### Example 4

### Three Drug Combinations (IR/IR/ER): Anti-Inflammatory/DPP-IV inhibitors/Biguanides

200 mg Ibuprofen or 250 mg Naproxen-Sodium or 325 mg of Aspirin or Aspirin-like drugs (e.g. Ascriptin, Ecotrin) or 50 mg to 400 mg Celecoxib or 200 mg Sulindac or an appropriate dose of other known anti-inflammatory drugs will be formulated in IR form in combination with anti-type II diabetes drugs such as 500 mg Metformin in an IR form with DPP-IV inhibitors such as 12.5 mg to 100 mg Sitagliptin, 3 mg to 25 mg Alogliptin or 2.5 mg Linagliptin in an IR form along with Biguanides such as 250 mg to 2000 mg Metformin in an ER form.

### Example 5

### Three Drug Combinations (IR/IR/ER): Anti-Inflammatory/SGLT-2 Inhibitors/Biguanides

200 mg Ibuprofen or 250 mg Naproxen-Sodium or 325 mg of Aspirin or Aspirin-like drugs (e.g. Ascriptin, Ecotrin) or 50 mg to 400 mg Celecoxib or 200 mg Sulindac or an appropriate dose of other known anti-inflammatory drugs will be formulated in IR form in combination with anti-type II diabetes drugs such as SGLT-2 (Sodium Glucose co-transporters) inhibitors such as 2.5 mg to 10 mg Dapagliflozin or 50 to 300 mg Canagliflozin formulated in an IR form along with Biguanides such as 500 mg Metformin in an ER form.

### Example 6

### Three Drug Combinations (IR/IR/DR): Anti-Inflammatory/DPP-IV inhibitors/SGLT-2 inhibitors

200 mg Ibuprofen or 250 mg Naproxen-Sodium or 325 mg of Aspirin or Aspirin-like drugs (e.g. Ascriptin, Ecotrin) or 50 mg to 400 mg Celecoxib or 200 mg Sulindac or an appropriate dose of other known anti-inflammatory drugs will be formulated in IR form in combination with anti-type II diabetes drugs such as 500 mg Metformin in an IR form with DPPIV inhibitors such as 12.5 mg to 100 mg Sitagliptin, 3 mg to 25 mg Alogliptin or 2.5 mg Linagliptin in an IR form along with SGLT-2 (Sodium Glucose co-transporters) inhibitors like 5 mg Dapagliflozin or 100 mg Canagliflozin in a DR form administered once or twice a day (b.i.d).

### Example 7

### Four Drug Combinations (IR/IR/ER/DR): Anti-Inflammatory/DPP-IV Inhibitors/Biguanides/SGLT-2 Inhibitors

200 mg Ibuprofen or 250 mg Naproxen-S odium or 325 mg of Aspirin or Aspirin-like drugs (e.g. Ascriptin, Ecotrin) or 50 mg to 400 mg Celecoxib or 200 mg Sulindac or an appropriate dose of other known anti-inflammatory drugs will be formulated in IR form with anti-type II diabetes drugs such as DPP-IV inhibitors such as 12.5 mg to 100 mg Sitagliptin, 3 mg to 25 mg Alogliptin or 2.5 mg Linagliptin in an IR form in combination with Biguanides such as 500 mg Metformin in an ER form along with SGLT-2 (Sodium Glucose co-transporters) inhibitors like 5 mg Dapagliflozin or 100 mg Canagliflozin in a DR form administered once or twice a day (b.i.d).

### Example 8

### Combination of 2-4 Drug Combinations with Incretin Mimetics

Two to four drug combinations described above can be combined with sub-cutaneous administration (S.C.) Incretin mimetics such as 5 ug Exenatide or 0.65 mg-1.8 mg Liraglutide (Victoza)

### Example 9

### Combination of 2-4 Drug Combinations with TZDs (Glitazones)

Two drug combinations (IR/DR): Anti-inflammatory/glitazones; 200 mg Ibuprofen or 250 mg Naproxen-Sodium or 325 mg of Aspirin or Aspirin-like drugs (e.g. Ascriptin, Ecotrin) or 50 mg to 400 mg Celecoxib or 200 mg Sulindac or an appropriate dose of other known anti-inflammatory drugs will be formulated in an IR form in combination with anti-type 2 diabetes drugs such as 30 mg Pioglitazone or 8 mg Rosiglitazone in a DR form.

Three drug combinations (IR/IR/DR): Anti-inflammatory/Biguanides/TZDs (Thiozolidinediones) or glitazones; 200 mg Ibuprofen or 250 mg Naproxen-Sodium or 325 mg of Aspirin or Aspirin-like drugs (e.g. Ascriptin, Ecotrin) or 50 mg to 400 mg Celecoxib or 200 mg Sulindac or an appropriate dose of other known anti-inflammatory drugs will be formulated in an IR form in combination with anti-type 2 diabetes drugs such as 500 mg to 1000 mg Metformin in an IR form with TZDs such as 30 mg Pioglitazone or 8 mg Rosiglitazone in a DR form.

Three drug combinations (IR/IR/DR): Anti-inflammatory/SGLT-2 inhibitors/glitazones; 200 mg Ibuprofen or 250 mg Naproxen-S odium or 325 mg of Aspirin or Aspirin-like drugs (e.g. Ascriptin, Ecotrin) or 50 mg to 400 mg Celecoxib or 200 mg Sulindac or an appropriate dose of other known anti-inflammatory drugs will be formulated in an IR form in combination with anti-type 2 diabetes drugs such as 25 mg Dapagliflozin or 300 mg Canagliflozin in an IR form with TZDs such as 30 mg Pioglitazone or 8 mg Rosiglitazone in a DR form.

Three drug combinations (IR/IR/DR): Anti-inflammatory/DPP-IV inhibitors/glitazones; 200 mg Ibuprofen or 250 mg Naproxen-S odium or 325 mg of Aspirin or Aspirin-like drugs (e.g. Ascriptin, Ecotrin) or 50 mg to 400 mg Celecoxib or 200 mg Sulindac or an appropriate dose of other known anti-inflammatory drugs will be formulated in an IR form in combination with anti-type 2 diabetes drugs such as 250 mg Sitagliptin or 25 mg Alogliptin or 10 mg Linagliptin in an IR form with TZDs such as 30 mg Pioglitazone or 8 mg Rosiglitazone in a DR form.

Four drug combinations (IR/IR/ER/DR): Anti-inflammatory/SGLT-2 inhibitors/Biguanides/Glitazones; 200 mg Ibuprofen or 250 mg Naproxen-Sodium or 325 mg of Aspirin or Aspirin-like drugs (e.g. Ascriptin, Ecotrin) or 50 mg to 400 mg Celecoxib or 200 mg Sulindac or an appropriate dose of other known anti-inflammatory drugs will be formulated in an IR form in combination with anti-type 2 diabetes drugs such as 25 mg Dapagliflozin or 300 mg Canagliflozin in an IR form with 500 mg to 1000 mg Metformin in an ER form TZDs such as 30 mg Pioglitazone or 8 mg Rosiglitazone in a DR form.

Four drug combinations (IR/IR/ER/DR): Anti-inflammatory/DPP-IV inhibitors/Biguanides/Glitazones; 200 mg Ibuprofen or 250 mg Naproxen-Sodium or 325 mg of Aspirin or Aspirin-like drugs (e.g. Ascriptin, Ecotrin) or 50 mg to 400 mg Celecoxib or 200 mg Sulindac or an appropriate dose of other known anti-inflammatory drugs will be formulated in an IR form in combination with anti-type 2 diabetes drugs such as 250 mg Sitagliptin or 25 mg Alogliptin or 10 mg Linagliptin in an IR form with 500 mg to 1000 mg Metformin in an ER form TZDs such as 30 mg Pioglitazone or 8 mg Rosiglitazone in a DR form.

### Example 10

### Lipid Lowering Drugs: Two Drug Combinations (IR/IR)

200 mg Ibuprofen or 250 mg Naproxen-S odium or 325 mg of Aspirin or Aspirin-like drugs (e.g. Ascriptin, Ecotrin) or 50 mg to 400 mg Celecoxib or 200 mg Sulindac or an appropriate dose of other known anti-inflammatory drugs will be formulated in IR form with Statins such as 5 mg-80 mg Atorvastatin or 10 mg to 80 mg Fluvastatin or 5 mg to 80 mg Lovastatin or 20 mg to 80 mg Provastatin or 2.5 mg to 40 mg Rosuvastatin or 2.5 mg to 40 mg Simvastatin; or Nicotinic acids (Niacin) such as 250 mg to 500 mg Nicolar or 250 mg to 500 mg Niaspan; or Fibrates such as 24 to 145 mg Tricor or 500 mg to 1200 mg Lopid or 2.5 mg to 10 mg Ezetimide and administered once or twice daily depending on patients response and tolerance.

### Example 11

### Lipid Lowering Drugs: Two Drug Combinations (IR/ER)

200 mg Ibuprofen or 250 mg Naproxen-S odium or 325 mg of Aspirin or Aspirin-like drugs (e.g. Ascriptin, Ecotrin) or 50 mg to 400 mg Celecoxib or 200 mg Sulindac or an appropriate dose of other known anti-inflammatory drugs will be formulated in IR form with Statins such as 80 mg Atorvastatin or 80 mg Provastatin or 40 mg Rosuvastatin or 40 mg Simvastatin; or Niacins such as 500 mg to 2000 mg Nicolar or 500 mg to 2000 mg Niaspan; Fibrates such as 145 mg Tricor or 600 mg to 1200 mg Lopid formulated in the ER form.

### Example 12

### Lipid Lowering Drugs: Three Drug Combinations (IR/IR/ER): Anti-Inflammatory/Statins/Niacin

200 mg Ibuprofen or 250 mg Naproxen-S odium or 325 mg of Aspirin or Aspirin-like drugs (e.g. Ascriptin, Ecotrin) or 50 mg to 400 mg Celecoxib or 200 mg Sulindac or an appropriate dose of other known anti-inflammatory drugs will be formulated in IR form with Statins such as 5 mg-80 mg Atorvastatin or 10 mg to 80 mg Fluvastatin or 5 mg to 80 mg Lovastatin or 20 mg to 80 mg Provastatin or 2.5 mg to 40 mg Rosuvastatin or 2.5 mg to 40 mg Simvastatin formulated in the IR form will be combined with Nicotinic acids such as 500 mg to 2000 mg Nicolar or 500 mg to 2000 mg Niaspan formulated in the ER form.

### Example 13

### Lipid Lowering Drugs: Three Drug Combinations (IR/IR/ER): Anti-Inflammatory/Statins/Fibrates

200 mg Ibuprofen or 250 mg Naproxen-Sodium or 325 mg of Aspirin or Aspirin-like drugs (e.g. Ascriptin, Ecotrin) or 50 mg to 400 mg Celecoxib or 200 mg Sulindac or an appropriate dose of other known anti-inflammatory drugs will be formulated in IR form with Statins such as 5 mg-80 mg Atorvastatin or 10 mg to 80 mg Fluvastatin or 5 mg to 80 mg Lovastatin or 20 mg to 80 mg Provastatin or 2.5 mg to 40 mg Rosuvastatin or 2.5 mg to 40 mg Simvastatin formulated in the IR form will be combined with Fibrates such as 145 mg Tricor or 600 mg to 1200 mg Lopid formulated in the ER form.

### Example 14

### Lipid Lowering Drugs: Three Drug Combinations (IR/IR/ER): Anti-Inflammatory/Niacins/Statins

200 mg Ibuprofen or 250 mg Naproxen-S odium or 325 mg of Aspirin or Aspirin-like drugs (e.g. Ascriptin, Ecotrin) or 50 mg to 400 mg Celecoxib or 200 mg Sulindac or an appropriate dose of other known anti-inflammatory drugs will be formulated in IR form with Niacins such as 500 mg to 2000 mg Nicolar or 500 mg to 2000 mg Niaspan formulated in the IR form will be combined with Statins such as 80 mg Atorvastatin or 80 mg Provastatin or 40 mg Rosuvastatin or 40 mg Simvastatin formulated in the ER form.

### Example 15

### Lipid Lowering Drugs: Three Drug Combinations (IR/IR/DR): Anti-Inflammatory/Fibrates/Statins

200 mg Ibuprofen or 250 mg Naproxen-S odium or 325 mg of Aspirin or Aspirin-like drugs (e.g. Ascriptin, Ecotrin) or 50 mg to 400 mg Celecoxib or 200 mg Sulindac or an appropriate dose of other known anti-inflammatory drugs will be formulated in IR form with Fibrates such as 24 to 145 mg Tricor or 500 mg to 1200 mg Lopid or 2.5 mg to 10 mg Ezetimide formulated in the IR form will be combined with Statins such as 80 mg Atorvastatin or 80 mg Provastatin or 40 mg Rosuvastatin or 40 mg Simvastatin formulated in the ER form.

### Example 16

### Lipid Lowering Drugs: Four Drug Combinations (IR/IR/ER/DR): Anti-Inflammatory/Statins/Niacins/Fibrates

200 mg Ibuprofen or 250 mg Naproxen-S odium or 325 mg of Aspirin or Aspirin-like drugs (e.g. Ascriptin, Ecotrin) or 50 mg to 400 mg Celecoxib or 200 mg Sulindac or an appropriate dose of other known anti-inflammatory drugs will be formulated in IR form with Statins such as 5 mg-80 mg Atorvastatin or 10 mg to 80 mg Fluvastatin or 5 mg to 80 mg Lovastatin or 20 mg to 80 mg Provastatin or 2.5 mg to 40 mg Rosuvastatin or 2.5 mg to 40 mg Simvastatin formulated in the IR form with Niacins such as 500 mg to 2000 mg Nicolar or 500 mg to 2000 mg Niaspan formulated in the ER form will be combined with Fibrates such as 24 to 145 mg Tricor or 500 mg to 1200 mg Lopid or 2.5 mg to 10 mg Ezetimide formulated in the DR form.

### Example 17

### Anti-Inflammatory/Anti-Hypertensive Drugs: Two Drug Combinations (IR/IR)

200 mg Ibuprofen or 250 mg Naproxen-S odium or 325 mg of Aspirin or Aspirin-like drugs (e.g. Ascriptin, Ecotrin) or 50 mg to 400 mg Celecoxib or 200 mg Sulindac or an appropriate dose of other known anti-inflammatory drugs will be formulated in IR form with Diuretics such as 12.5 mg to 400 mg Spiranolactone or 25 mg to 100 mg Triampterene or 12.5 mg to 100 mg Hydrochlorothiazide (HCTZ) or 7.5 mg to 100 mg Chlorthalidone or 10 mg to 600 mg Furosemide or 0.5 mg to 5 mg Amiloride Hydrochloride or 0.25 mg to 2.5 mg Metolazone; or Beta blockers such as 0.5 mg to 60 mg Timolol or 1.25 mg to 10 mg Cartelol Hydrochloride or 1.5 mg to 50 mg Corredilol or 20 mg to 640 mg Propranodol or 2.5 mg to 40 mg Betaxolol or 10 mg to 80 mg Penbutolol Sulfate or 50 mg to 450 mg Metoprolol or 100 mg to 1200 mg Acebutolol or 25 mg to 200 mg Atenolol or 2.5 mg to 20 mg Pindolol or 50 mg to 2400 mg Labetolol; Alpha blockers such as 0.5 mg to 16 mg Doxazocin or 0.5 mg to 20 mg Terazocin or 0.5 mg to 20 mg Prazocin; Angiotensin-converting enzyme (ACE) inhibitors such as 0.5 mg to 80 mg Quinapril or 1.25 mg to 20 mg Ramipril or 12.5 mg to 450 mg Captopril or 0.5 mg to 8 mg Trandolapril or 5 mg to 40 mg Benazepril or 5 mg to 80 mg Fosinopril or 0.5 mg to 80 mg Lisinopril or 5 mg to 60 mg Moexipril or 1.25 mg to 40 mg Enalapril; or Angiotensin II Receptor Blockers (ARBs) such as 2 mg to 32 mg Candesartan or 75 mg to 300 mg Irbesartan or 10 mg to 40 mg Olmesartan or 12.5 mg to Losartan or 40 mg to 320 mg Valsartan or 10 mg to 80 mg Telmisartan or 200 mg to 800 mg Eprosartan; or Calcium Channel Blockers (CCBs) such as 15 mg to 60 mg Nifedipine or 15 mg to 60 mg Diltiazem or 1.25 mg to 10 mg Amlodipine or 1.25 mg to 60 mg of other CCBs (e.g. Verampil, Nicardipine, Isradipine, Felodipine or Nisoldipine); Central Agonists such as 125 mg to 2000 mg Methyldopa or 0.05 mg to 2.4 mg Clonidine or 0.5 mg to 3 mg Guanfacine or 2 mg to 32 mg Guanbenz; or Peripheral-acting Adrenergic blockers such as 5 mg to 75 mg Guanadrel or 5 mg to 50 mg Guanethidine or 0.05 mg to 1 mg Reserpine; or Direct Vasodilators such as 5 mg to 300 mg Hydralazine or 2.5 mg to 100 mg Minoxidil; or Direct Renin Inhibitors such as 75 mg to 300 mg Tekturana formulated in the IR form.

### Example 18

### Anti-Inflammatory/Anti-Hypertensive Drugs: Two Drug Combinations (IR/ER)

200 mg Ibuprofen or 250 mg Naproxen-S odium or 325 mg of Aspirin or Aspirin-like drugs (e.g. Ascriptin, Ecotrin) or 50 mg to 400 mg Celecoxib or 200 mg Sulindac or an appropriate dose of other known anti-inflammatory drugs will be formulated in IR form with Diuretics such as 200 mg to 400 mg Spiranolactone or 50 mg to 100 mg Triampterene or 50 mg to 100 mg Hydrochlorothiazide (HCTZ) or 50 mg to 100 mg Chlorthalidone or 100 mg to 600 mg Furosemide or 2.5 mg to 5 mg Amiloride Hydrochloride or 1 mg to 2.5 mg Metolazone; or Beta blockers such as 10 mg to 60 mg Timolol or 2.5 mg to 10 mg Cartelol Hydrochloride or 5 mg to 50 mg Corredilol or 100 mg to 640 mg Propranolol or 10 mg to 40 mg Betaxolol or 50 mg to 80 mg Penbutolol Sulfate or 250 mg to 450 mg Metoprolol or 250 mg to 1200 mg Acebutolol or 100 mg to 200 mg Atenolol or 10 mg to 20 mg Pindolol or 250 mg to 2400 mg Labetolol; Alpha blockers such as 2.5 mg to 16 mg Doxazocin or 2.5 mg to 20 mg Terazocin or 2.5 mg to 20 mg Prazocin; Angiotensin-converting enzyme (ACE) inhibitors such as 5 mg to 80 mg Quinapril or 2.5 mg to 20 mg Ramipril or 50 mg to 450 mg Captopril or 2.5 mg to 8 mg Trandolapril or 15 mg to 40 mg Benazepril or 25 mg to 80 mg Fosinopril or 5 mg to 80 mg Lisinopril or 15 mg to 60 mg Moexipril or 5 mg to 40 mg Enalapril; or Angiotensin II Receptor Blockers (ARBs) such as 10 mg to 32 mg Candesartan or 150 mg to 300 mg Irbesartan or 25 mg to 40 mg Olmesartan or 25 mg to Losartan or 100 mg to 320 mg Valsartan or 25 mg to 80 mg Telmisartan or 250 mg to 800 mg Eprosartan; or Calcium Channel Blockers (CCBs) such as 25 mg to 60 mg Nifedipine or 25 mg to 60 mg Diltiazem or 5.0 mg to 10 mg Amlodipine or 5 mg to 60 mg of other CCBs (e.g. Verampil, Nicardipine, Isradipine, Felodipine or Nisoldipine); Central Agonists such as 250 mg to 2000 mg Methyldopa or 0.1 mg to 2.4 mg Clonidine or 1.0 mg to 3 mg Guanfacine or 5 mg to 32 mg Guanabenz; or Peripheral-acting Adrenergic blockers such as 10 mg to 75 mg Guanadrel or 10 mg to 50 mg Guanethidine or 0.05 mg to 1 mg Reserpine; or Direct Vasodilators such as 25 mg to 300 mg Hydralazine or 5 mg to 100 mg Minoxidil; or Direct Renin Inhibitors such as 100 mg to 300 mg Tekturana formulated in the ER form.

### Example 19

### Three Drug Combinations (IR/IR/ER): Anti-Inflammatory/Diuretics/B Eta Blockers

200 mg Ibuprofen or 250 mg Naproxen-S odium or 325 mg of Aspirin or Aspirin-like drugs (e.g. Ascriptin, Ecotrin) or 50 mg to 400 mg Celecoxib or 200 mg Sulindac or an appropriate dose of other known anti-inflammatory drugs will be formulated in IR form with Diuretics such as 12.5 mg to 400 mg Spiranolactone or 25 mg to 100 mg Triampterene or 12.5 mg to 100 mg Hydrochlorothiazide (HCTZ) or 7.5 mg to 100 mg Chlorthalidone or 10 mg to 600 mg Furosemide or 0.5 mg to 5 mg Amiloride Hydrochloride or 0.25 mg to 2.5 mg Metolazone formulated in IR form combined with Beta blockers such as 10 mg to 60 mg Timolol or 2.5 mg to 10 mg Cartelol Hydrochloride or 5 mg to 50 mg Corredilol or 100 mg to 640 mg Propranolol or 10 mg to 40 mg Betaxolol or 50 mg to 80 mg Penbutolol Sulfate or 250 mg to 450 mg Metoprolol or 250 mg to 1200 mg Acebutolol or 100 mg to 200 mg Atenolol or 10 mg to 20 mg Pindolol or 250 mg to 2400 mg Labetolol formulated in ER form.

### Example 20

### Three Drug Combinations (IR/IR/ER): Anti-Inflammatory/Beta Blockers/Diuretics

200 mg Ibuprofen or 250 mg Naproxen-S odium or 325 mg of Aspirin or Aspirin-like drugs (e.g. Ascriptin, Ecotrin) or 50 mg to 400 mg Celecoxib or 200 mg Sulindac or an appropriate dose of other known anti-inflammatory drugs will be formulated in IR form with Beta blockers such as 0.5 mg to 60 mg Timolol or 1.25 mg to 10 mg Cartelol Hydrochloride or 1.5 mg to 50 mg Corredilol or 20 mg to 640 mg Propranolol or 2.5 mg to 40 mg Betaxolol or 10 mg to 80 mg Penbutolol Sulfate or 50 mg to 450 mg Metoprolol or 100 mg to 1200 mg Acebutolol or 25 mg to 200 mg Atenolol or 2.5 mg to 20 mg Pindolol or 50 mg to 2400 mg Labetolol formulated in the IR form and combined with Diuretics such as 200 mg to 400 mg Spiranolactone or 50 mg to 100 mg Triampterene or 50 mg to 100 mg Hydrochlorothiazide (HCTZ) or 50 mg to 100 mg Chlorthalidone or 100 mg to 600 mg Furosemide or 2.5 mg to 5 mg Amiloride Hydrochloride or 1 mg to 2.5 mg Metolazone formulated in the ER form.

### Example 21

### Three Drug Combinations (IR/IR/ER): Anti-Inflammatory/Diuretics/a CE Inhibitors

200 mg Ibuprofen or 250 mg Naproxen-Sodium or 325 mg of Aspirin or Aspirin-like drugs (e.g. Ascriptin, Ecotrin) or 50 mg to 400 mg Celecoxib or 200 mg Sulindac or an appropriate dose of other known anti-inflammatory drugs will be formulated in IR form with Diuretics such as 12.5 mg to 400 mg Spiranolactone or 25 mg to 100 mg Triampterene or 12.5 mg to 100 mg Hydrochlorothiazide (HCTZ) or 7.5 mg to 100 mg Chlorthalidone or 10 mg to 600 mg Furosemide or 0.5 mg to 5 mg Amiloride Hydrochloride or 0.25 mg to 2.5 mg Metolazone formulated in IR form will be combined with ACE inhibitors such as 5 mg to 80 mg Quinapril or 2.5 mg to 20 mg Ramipril or 50 mg to 450 mg Captopril or 2.5 mg to 8 mg Trandolapril or 15 mg to 40 mg Benazepril or 25 mg to 80 mg Fosinopril or 5 mg to 80 mg Lisinopril or 15 mg to 60 mg Moexipril or 5 mg to 40 mg Enalapril formulated in the form of ER.

### Example 22

### Three Drug Combinations (IR/IR/ER): Anti-Inflammatory/ACE Inhibitors/Diuretics

200 mg Ibuprofen or 250 mg Naproxen-S odium or 325 mg of Aspirin or Aspirin-like drugs (e.g. Ascriptin, Ecotrin) or 50 mg to 400 mg Celecoxib or 200 mg Sulindac or an appropriate dose of other known anti-inflammatory drugs will be formulated in IR form with ACE inhibitors such as 0.5 mg to 80 mg Quinapril or 1.25 mg to 20 mg Ramipril or 12.5 mg to 450 mg Captopril or 0.5 mg to 8 mg Trandolapril or 5 mg to 40 mg Benazepril or 5 mg to 80 mg Fosinopril or 0.5 mg to 80 mg Lisinopril or 5 mg to 60 mg Moexipril or 1.25 mg to 40 mg Enalapril formulated in the IR form will be combined with Diuretics such as 12.5 mg to 400 mg Spiranolactone or 25 mg to 100 mg Triampterene or 12.5 mg to 100 mg Hydrochlorothiazide (HCTZ) or 7.5 mg to 100 mg Chlorthalidone or 10 mg to 600 mg Furosemide or 0.5 mg to 5 mg Amiloride Hydrochloride or 0.25 mg to 2.5 mg Metolazone formulated in the form of ER.

### Example 23

### Three Drug Combinations (IR/IR/ER): Anti-Inflammatory/Diuretics/ARBs

200 mg Ibuprofen or 250 mg Naproxen-Sodium or 325 mg of Aspirin or Aspirin-like drugs (e.g. Ascriptin, Ecotrin) or 50 mg to 400 mg Celecoxib or 200 mg Sulindac or an appropriate dose of other known anti-inflammatory drugs will be formulated in the IR form with Diuretics such as 12.5 mg to 400 mg Spiranolactone or 25 mg to 100 mg Triampterene or 12.5 mg to 100 mg Hydrochlorothiazide (HCTZ) or 7.5 mg to 100 mg Chlorthalidone or 10 mg to 600 mg Furosemide or 0.5 mg to 5 mg Amiloride Hydrochloride or 0.25 mg to 2.5 mg Metolazone formulated in IR form will be combined with ARBs such as 2 mg to 32 mg Candesartan or 75 mg to 300 mg Irbesartan or 10 mg to 40 mg Olmesartan or 12.5 mg to Losartan or 40 mg to 320 mg Valsartan or 10 mg to 80 mg Telmisartan or 200 mg to 800 mg Eprosartan formulated in the ER form.

### Example 24

### Three Drug Combinations (IR/IR/ER): Anti-Inflammatory/ARBs/Diuretics

200 mg Ibuprofen or 250 mg Naproxen-S odium or 325 mg of Aspirin or Aspirin-like drugs (e.g. Ascriptin, Ecotrin) or 50 mg to 400 mg Celecoxib or 200 mg Sulindac or an appropriate dose of other known anti-inflammatory drugs will be formulated in the IR form with (ARBs) such as 2 mg to 32 mg Candesartan or 75 mg to 300 mg Irbesartan or 10 mg to 40 mg Olmesartan or 12.5 mg to Losartan or 40 mg to 320 mg Valsartan or 10 mg to 80 mg Telmisartan or 200 mg to 800 mg Eprosartan formulated in the IR form will be combined with Diuretics such as 12.5 mg to 400 mg Spiranolactone or 25 mg to 100 mg Triampterene or 12.5 mg to 100 mg Hydrochlorothiazide (HCTZ) or 7.5 mg to 100 mg Chlorthalidone or 10 mg to 600 mg Furosemide or 0.5 mg to 5 mg Amiloride Hydrochloride or 0.25 mg to 2.5 mg Metolazone formulated in the form of ER.

### Example 25

### Three Drug Combinations (IR/IR/ER): Anti-Inflammatory/Diuretics/C CBs

200 mg Ibuprofen or 250 mg Naproxen-S odium or 325 mg of Aspirin or Aspirin-like drugs (e.g. Ascriptin, Ecotrin) or 50 mg to 400 mg Celecoxib or 200 mg Sulindac or an appropriate dose of other known anti-inflammatory drugs will be formulated in the IR form with Diuretics such as 12.5 mg to 400 mg Spiranolactone or 25 mg to 100 mg Triampterene or 12.5 mg to 100 mg Hydrochlorothiazide (HCTZ) or 7.5 mg to 100 mg Chlorthalidone or 10 mg to 600 mg Furosemide or 0.5 mg to 5 mg Amiloride Hydrochloride or 0.25 mg to 2.5 mg Metolazone formulated in IR form will be combined with (CCBs) such as 25 mg to 60 mg Nifedipine or 25 mg to 60 mg Diltiazem or 5.0 mg to 10 mg Amlodipine or 5 mg to 60 mg of other CCBs (e.g. Verampil, Nicardipine, Isradipine, Felodipine or Nisoldipine) formulated in the ER form.

### Example 26

### Three Drug Combinations (IR/IR/ER): Anti-Inflammatory/CCBs/Diuretics

200 mg Ibuprofen or 250 mg Naproxen-S odium or 325 mg of Aspirin or Aspirin-like drugs (e.g. Ascriptin, Ecotrin) or 50 mg to 400 mg Celecoxib or 200 mg Sulindac or an appropriate dose of other known anti-inflammatory drugs will be formulated in the IR form with CCBs such as 15 mg to 60 mg Nifedipine or 15 mg to 60 mg Diltiazem or 1.25 mg to 10 mg Amlodipine or 1.25 mg to 60 mg of other CCBs (e.g. Verampil, Nicardipine, Isradipine, Felodipine or Nisoldipine) will be combined with Diuretics such as 12.5 mg to 400 mg Spiranolactone or 25 mg to 100 mg Triampterene or 12.5 mg to 100 mg Hydrochlorothiazide (HCTZ) or 7.5 mg to 100 mg Chlorthalidone or 10 mg to 600 mg Furosemide or 0.5 mg to 5 mg Amiloride Hydrochloride or 0.25 mg to 2.5 mg Metolazone formulated in the form of ER.

### Example 27

### Three Drug Combinations (IR/IR/ER): Anti-Inflammatory/Diuretics/Central Agonists

200 mg Ibuprofen or 250 mg Naproxen-S odium or 325 mg of Aspirin or Aspirin-like drugs (e.g. Ascriptin, Ecotrin) or 50 mg to 400 mg Celecoxib or 200 mg Sulindac or an appropriate dose of other known anti-inflammatory drugs will be formulated in the IR form with Diuretics such as 12.5 mg to 400 mg Spiranolactone or 25 mg to 100 mg Triampterene or 12.5 mg to 100 mg Hydrochlorothiazide (HCTZ) or 7.5 mg to 100 mg Chlorthalidone or 10 mg to 600 mg Furosemide or 0.5 mg to 5 mg Amiloride Hydrochloride or 0.25 mg to 2.5 mg Metolazone formulated in IR form will be combined with Central Agonists such as 250 mg to 2000 mg Methyldopa or 0.1 mg to 2.4 mg Clonidine or 1.0 mg to 3 mg Guanfacine or 5 mg to 32 mg Guanabenz formulated in the ER form.

### Example 28

### Three Drug Combinations (IR/IR/ER): Anti-Inflammatory/Central Agonists/Diuretics

200 mg Ibuprofen or 250 mg Naproxen-S odium or 325 mg of Aspirin or Aspirin-like drugs (e.g. Ascriptin, Ecotrin) or 50 mg to 400 mg Celecoxib or 200 mg Sulindac or an appropriate dose of other known anti-inflammatory drugs will be formulated in the IR form with Central Agonists such as 125 mg to 2000 mg Methyldopa or 0.05 mg to 2.4 mg Clonidine or 0.5 mg to 3 mg Guanfacine or 2 mg to 32 mg Guanabenz formulated in the IR form will be combined with Diuretics such as 12.5 mg to 400 mg Spiranolactone or 25 mg to 100 mg Triampterene or 12.5 mg to 100 mg Hydrochlorothiazide (HCTZ) or 7.5 mg to 100 mg Chlorthalidone or 10 mg to 600 mg Furosemide or 0.5 mg to 5 mg Amiloride Hydrochloride or 0.25 mg to 2.5 mg Metolazone formulated in the form of ER.

### Example 29

### Three Drug Combinations (IR/IR/ER): Anti-Inflammatory/Peripheral-Acting Adrenergic Blockers/Diuretics

200 mg Ibuprofen or 250 mg Naproxen-S odium or 325 mg of Aspirin or Aspirin-like drugs (e.g. Ascriptin, Ecotrin) or 50 mg to 400 mg Celecoxib or 200 mg Sulindac or an appropriate dose of other known anti-inflammatory drugs will be formulated in the IR form with Peripheral-acting Adrenergic blockers such as 10 mg to 75 mg Guanadrel or 10 mg to 50 mg Guanethidine or 0.05 mg to 1 mg Reserpine formulated in the IR form will be combined with Diuretics such as 200 mg to 400 mg Spiranolactone or 50 mg to 100 mg Triampterene or 50 mg to 100 mg Hydrochlorothiazide (HCTZ) or 50 mg to 100 mg Chlorthalidone or 100 mg to 600 mg Furosemide or 2.5 mg to 5 mg Amiloride Hydrochloride or 1 mg to 2.5 mg Metolazone formulated in the form of ER.

### Example 30

### Three Drug Combinations (IR/IR/ER): Anti-Inflammatory/Diuretics/Peripheral-Acting Adrenergic Blockers

200 mg Ibuprofen or 250 mg Naproxen-S odium or 325 mg of Aspirin or Aspirin-like drugs (e.g. Ascriptin, Ecotrin) or 50 mg to 400 mg Celecoxib or 200 mg Sulindac or an appropriate dose of other known anti-inflammatory drugs will be formulated in the IR form with Diuretics such as 12.5 mg to 400 mg Spiranolactone or 25 mg to 100 mg Triampterene or 12.5 mg to 100 mg Hydrochlorothiazide (HCTZ) or 7.5 mg to 100 mg Chlorthalidone or 10 mg to 600 mg Furosemide or 0.5 mg to 5 mg Amiloride Hydrochloride or 0.25 mg to 2.5 mg Metolazone formulated in IR form will be combined

With Peripheral-acting Adrenergic blockers such as 10 mg to 75 mg Guanadrel or 10 mg to 50 mg Guanethidine or 0.05 mg to 1 mg Reserpine formulated in the ER form.

### Example 31

### Three Drug Combinations (IR/IR/ER): Anti-Inflammatory/Diuretics/Direct Vasodilators

200 mg Ibuprofen or 250 mg Naproxen-S odium or 325 mg of Aspirin or Aspirin-like drugs (e.g. Ascriptin, Ecotrin) or 50 mg to 400 mg Celecoxib or 200 mg Sulindac or an appropriate dose of other known anti-inflammatory drugs will be formulated in the IR form with Diuretics such as 12.5 mg to 400 mg Spiranolactone or 25 mg to 100 mg Triampterene or 12.5 mg to 100 mg Hydrochlorothiazide (HCTZ) or 7.5 mg to 100 mg Chlorthalidone or 10 mg to 600 mg Furosemide or 0.5 mg to 5 mg Amiloride Hydrochloride or 0.25 mg to 2.5 mg Metolazone formulated in IR form will be combined with Direct Vasodilators such as 25 mg to 300 mg Hydralazine or 5 mg to 100 mg Minoxidil formulated in the ER form or direct vasodilators such as 25 mg to 300 mg Hydralazine or 5 mg to 100 mg Minoxidil formulated in the ER form.

### Example 32

### Three Drug Combinations (IR/IR/ER): Anti-Inflammatory/Direct Vasodilators/Diuretics

200 mg Ibuprofen or 250 mg Naproxen-Sodium or 325 mg of Aspirin or Aspirin-like drugs (e.g. Ascriptin, Ecotrin) or 50 mg to 400 mg Celecoxib or 200 mg Sulindac or an appropriate dose of other known anti-inflammatory drugs will be formulated in the IR form with Direct Vasodilators such as 5 mg to 300 mg Hydralazine or 2.5 mg to 100 mg Minoxidil formulated in the IR form will be combined with Diuretics such as 200 mg to 400 mg Spiranolactone or 50 mg to 100 mg Triampterene or 50 mg to 100 mg Hydrochlorothiazide (HCTZ) or 50 mg to 100 mg Chlorthalidone or 100 mg to 600 mg Furosemide or 2.5 mg to 5 mg Amiloride Hydrochloride or 1 mg to 2.5 mg Metolazone formulated in the form of ER.

### Example 33

### Three Drug Combinations (IR/IR/ER): Anti-Inflammatory/Diuretics/Direct Renin Inhibitors

200 mg Ibuprofen or 250 mg Naproxen-S odium or 325 mg of Aspirin or Aspirin-like drugs (e.g. Ascriptin, Ecotrin) or 50 mg to 400 mg Celecoxib or 200 mg Sulindac or an appropriate dose of other known anti-inflammatory drugs will be formulated in the IR form with Diuretics such as 12.5 mg to 400 mg Spiranolactone or 25 mg to 100 mg Triampterene or 12.5 mg to 100 mg Hydrochlorothiazide (HCTZ) or 7.5 mg to 100 mg Chlorthalidone or 10 mg to 600 mg Furosemide or 0.5 mg to 5 mg Amiloride Hydrochloride or 0.25 mg to 2.5 mg Metolazone formulated in IR form will be combined with Direct Renin Inhibitors such as 100 mg to 300 mg Tekturana formulated in the ER form.

### Example 34

### Three Drug Combinations (IR/IR/ER): Anti-Inflammatory/Direct Renin Inhibitors/Diuretics

200 mg Ibuprofen or 250 mg Naproxen-S odium or 325 mg of Aspirin or Aspirin-like drugs (e.g. Ascriptin, Ecotrin) or 50 mg to 400 mg Celecoxib or 200 mg Sulindac or an appropriate dose of other known anti-inflammatory drugs will be formulated in the IR form with Direct Renin Inhibitors such as 75 mg to 300 mg Tekturana formulated in the IR form will be combined with Diuretics such as 200 mg to 400 mg Spiranolactone or 50 mg to 100 mg Triampterene or 50 mg to 100 mg Hydrochlorothiazide (HCTZ) or 50 mg to 100 mg Chlorthalidone or 100 mg to 600 mg Furosemide or 2.5 mg to 5 mg Amiloride Hydrochloride or 1 mg to 2.5 mg Metolazone formulated in the form of ER.

### Example 35

### Four Drug Combinations (IR/IR/ER/DR): Anti-Inflammatory/Diuretic/CCB/ACE Inhibitor

200 mg Ibuprofen or 250 mg Naproxen-S odium or 325 mg of Aspirin or Aspirin-like drugs (e.g. Ascriptin, Ecotrin) or 50 mg to 400 mg Celecoxib or 200 mg Sulindac or an appropriate dose of other known anti-inflammatory drugs will be formulated in the IR form with Diuretics such as 12.5 mg to 400 mg Spiranolactone or 25 mg to 100 mg Triampterene or 12.5 mg to 100 mg Hydrochlorothiazide (HCTZ) or 7.5 mg to 100 mg Chlorthalidone or 10 mg to 600 mg Furosemide or 0.5 mg to 5 mg Amiloride Hydrochloride or 0.25 mg to 2.5 mg Metolazone formulated in the IR form with Calcium Channel Blockers (CCBs) such as 25 mg to 60 mg Nifedipine or 25 mg to 60 mg Diltiazem or 5.0 mg to 10 mg Amlodipine or 5 mg to 60 mg of other CCBs (e.g. Verampil, Nicardipine, Isradipine, Felodipine or Nisoldipine) formulated in the ER form will be combined with ACE inhibitors such as 0.5 mg to 80 mg Quinapril or 1.25 mg to 20 mg Ramipril or 12.5 mg to 450 mg Captopril or 0.5 mg to 8 mg Trandolapril or 5 mg to 40 mg Benazepril or 5 mg to 80 mg Fosinopril or 0.5 mg to 80 mg Lisinopril or 5 mg to 60 mg Moexipril or 1.25 mg to 40 mg Enalapril formulated in the DR form.

### Example 36

### Anti-Obesity: Two Drug Combinations (IR/IR)

200 mg Ibuprofen or 250 mg Naproxen-S odium or 325 mg of Aspirin or Aspirin-like drugs (e.g. Ascriptin, Ecotrin) or 50 mg to 400 mg Celecoxib or 200 mg Sulindac or an appropriate dose of other known anti-inflammatory drugs will be formulated in the IR form with 50 mg to 120 mg Xenical or 10 mg to 37.5 mg Phentermine or 5 mg to 10 mg Lorcaserin or 10 mg to 37.5 mg Phentermine formulated in the form of IR.

### Example 37

### Two Drug Combinations (IR/ER): Anti-Obesity/Anti-Inflammatory

50 mg to 120 mg Xenical or 10 mg to 37.5 mg Phentermine or 5 mg to 10 mg Lorcaserin or 10 mg to 37.5 mg Phentermine formulated in the form of IR will be combined with 250 mg 500 mg Ibuprofen or 300 mg to 500 mg Aspirin-like drugs or 200 mg to 400 mg Celecoxib or 200 mg to 500 mg of Sulindac or other known anti-inflammatory drugs will be formulated in the form of ER.

### Example 38

### Three Drug Combinations (IR/IR/ER): Anti-Inflammatory/Anti-Obesity/DPPIV Inhibitors

200 mg Ibuprofen or 250 mg Naproxen-S odium or 325 mg of Aspirin or Aspirin-like drugs (e.g. Ascriptin, Ecotrin) or 50 mg to 400 mg Celecoxib or 200 mg Sulindac or an appropriate dose of other known anti-inflammatory drugs will be formulated in the IR form with 50 mg to 120 mg Xenical or 10 mg to 37.5 mg Phentermine or 5 mg to 10 mg Lorcaserin or 10 mg to 37.5 mg Phentermine formulated in the form of IR will be combined with DPP-IV inhibitors such as 250 mg Sitagliptin, 25 mg Alogliptin or 10 mg Linagliptin formulated in the ER form.

### Example 39

### Three Drug Combinations (IR/IR/S.C. Injection): Anti-Inflammatory/Anti-Obesity/Incretin Mimetics

200 mg Ibuprofen or 250 mg Naproxen-S odium or 325 mg of Aspirin or Aspirin-like drugs (e.g. Ascriptin, Ecotrin) or 50 mg to 400 mg Celecoxib or 200 mg Sulindac or an appropriate dose of other known anti-inflammatory drugs will be formulated in the IR form with 50 mg to 120 mg Xenical or 10 mg to 37.5 mg Phentermine or 5 mg to 10 mg Lorcaserin or 10 mg to 37.5 mg Phentermine formulated in the form of IR will be combined with Subcutaneous (S.C.) injections of Incretin mimetics such as 5 ug Exenatide or 0.65 mg to 1.8 mg Liraglutide or 0.6 mg to 1.8 mg Victoza.

### Example 40

### Prediabetes or Prevention of on-Set of Type II Diabetes Drugs: Two Drug Combinations (IR/IR): Anti-Inflammatory/ARBs or ACE Inhibitors

200 mg Ibuprofen or 250 mg Naproxen-S odium or 325 mg of Aspirin or Aspirin-like drugs (e.g. Ascriptin, Ecotrin) or 50 mg to 400 mg Celecoxib or 200 mg Sulindac or an appropriate dose of other known anti-inflammatory drugs will be formulated in the IR form with (ACE) inhibitors such as 0.5 mg to 80 mg Quinapril or 1.25 mg to 20 mg Ramipril or 12.5 mg to 450 mg Captopril or 0.5 mg to 8 mg Trandolapril or 5 mg to 40 mg Benazepril or 5 mg to 80 mg Fosinopril or 0.5 mg to 80 mg Lisinopril or 5 mg to 60 mg Moexipril or 1.25 mg to 40 mg Enalapril; or Angiotensin II Receptor Blockers (ARBs) such as 2 mg to 32 mg Candesartan or 75 mg to 300 mg Irbesartan or 10 mg to 40 mg Olmesartan or 12.5 mg to Losartan or 40 mg to 320 mg Valsartan or 10 mg to 80 mg Telmisartan or 200 mg to 800 mg Eprosartan formulated in the IR form.

### Example 41

### Prediabetes or Prevention of on-Set of Type II Diabetes Drugs: Three Drug Combinations (IR/IR/ER): Anti-Inflammatory/ARBs/Metformin

200 mg Ibuprofen or 250 mg Naproxen-S odium or 325 mg of Aspirin or Aspirin-like drugs (e.g. Ascriptin, Ecotrin) or 50 mg to 400 mg Celecoxib or 200 mg Sulindac or an appropriate dose of other known anti-inflammatory drugs will be formulated in the IR form with (ARBs) such as 2 mg to 32 mg Candesartan or 75 mg to 300 mg Irbesartan or 10 mg to 40 mg Olmesartan or 12.5 mg to Losartan or 40 mg to 320 mg Valsartan or 10 mg to 80 mg Telmisartan or 200 mg to 800 mg Eprosartan formulated in the IR form will be combined with 500 mg to 2000 mg Metformin formulated in the ER form.

### Example 42

### Prediabetes or Prevent or Delay the on-Set of Type II Diabetes Drugs: Three Drug Combinations (IR/IR/ER): Anti-Inflammatory/ACE Inhibitors/Metformin

200 mg Ibuprofen or 250 mg Naproxen-Sodium or 325 mg of Aspirin or Aspirin-like drugs (e.g. Ascriptin, Ecotrin) or 50 mg to 400 mg Celecoxib or 200 mg Sulindac or an appropriate dose of other known anti-inflammatory drugs will be formulated in the IR form with ACE inhibitors such as 0.5 mg to 80 mg Quinapril or 1.25 mg to 20 mg Ramipril or 12.5 mg to 450 mg Captopril or 0.5 mg to 8 mg Trandolapril or 5 mg to 40 mg Benazepril or 5 mg to 80 mg Fosinopril or 0.5 mg to 80 mg Lisinopril or 5 mg to 60 mg Moexipril or 1.25 mg to 40 mg Enalapril formulated in the IR form will be combined with 500 mg to 2000 mg Metformin formulated in the ER form.

### Example 43

### Prediabetes or Prevention of on-Set of Type II Diabetes Drugs: Four Drug Combinations (IR/IR/ER/DR): Anti-Inflammatory/ARBs/Metformin/ACE Inhibitor

200 mg Ibuprofen or 250 mg Naproxen-S odium or 325 mg of Aspirin or Aspirin-like drugs (e.g. Ascriptin, Ecotrin) or 50 mg to 400 mg Celecoxib or 200 mg Sulindac or an appropriate dose of other known anti-inflammatory drugs will be formulated in the IR form with (ARBs) such as 2 mg to 32 mg Candesartan or 75 mg to 300 mg Irbesartan or 10 mg to 40 mg Olmesartan or 12.5 mg to Losartan or 40 mg to 320 mg Valsartan or 10 mg to 80 mg Telmisartan or 200 mg to 800 mg Eprosartan formulated in the IR form with 500 mg to 2000 mg Metformin formulated in the ER form will be combined with ACE inhibitors such as 0.5 mg to 80 mg Quinapril or 1.25 mg to 20 mg Ramipril or 12.5 mg to 450 mg Captopril or 0.5 mg to 8 mg Trandolapril or 5 mg to 40 mg Benazepril or 5 mg to 80 mg Fosinopril or 0.5 mg to 80 mg Lisinopril or 5 mg to 60 mg Moexipril or 1.25 mg to 40 mg Enalapril formulated in the DR form.

### Example 44

### FDC Formulations for Clinically Managing Insulin Resistance Syndrome or Metabolic Syndrome: Two Drug Combinations (IR/IR/ER): Anti-Inflammatory/Metformin/Metformin

200 mg Ibuprofen or 250 mg Naproxen-S odium or 325 mg of Aspirin or Aspirin-like drugs (e.g. Ascriptin, Ecotrin) or 50 mg to 400 mg Celecoxib or 200 mg Sulindac or an appropriate dose of other known anti-inflammatory drugs will be formulated in the IR form with 250 mg to 500 mg Metformin formulated in the IR form will be combined with 250 mg to 2000 mg Metformin formulated in the ER form.

### Example 45

### FDC Formulations for Clinically Managing Insulin Resistance Syndrome or Metabolic Syndrome: Two Drug Combinations (IR/IR/DR): Anti-Inflammatory/Metformin/Metformin

200 mg Ibuprofen or 250 mg Naproxen-S odium or 325 mg of Aspirin or Aspirin-like drugs (e.g. Ascriptin, Ecotrin) or 50 mg to 400 mg Celecoxib or 200 mg Sulindac or an appropriate dose of other known anti-inflammatory drugs will be formulated in the IR form with 250 mg to 500 mg Metformin formulated in the IR form will be combined with 250 mg to 2000 mg Metformin formulated in the DR form.

### Example 46

### FDC Formulations for Clinically Managing Insulin Resistance Syndrome or Metabolic Syndrome: Three Drug Combinations (IR/IR/ER): Anti-Inflammatory/Metformin/Atorvastatin

200 mg Ibuprofen or 250 mg Naproxen-S odium or 325 mg of Aspirin or Aspirin-like drugs (e.g. Ascriptin, Ecotrin) or 50 mg to 400 mg Celecoxib or 200 mg Sulindac or an appropriate dose of other known anti-inflammatory drugs will be formulated in the IR form with 250 mg to 500 mg Metformin formulated in the IR form will be combined with Statins such as 80 mg Atorvastatin or 80 mg Provastatin or 40 mg Rosuvastatin or 40 mg Simvastatin formulated in the ER form.

### Example 47

### FDC Formulations for Clinically Managing Insulin Resistance Syndrome or Metabolic Syndrome: Three Drug Combinations (IR/IR/ER): Anti-Inflammatory/Metformin/ACE Inhibitors

200 mg Ibuprofen or 250 mg Naproxen-S odium or 325 mg of Aspirin or Aspirin-like drugs (e.g. Ascriptin, Ecotrin) or 50 mg to 400 mg Celecoxib or 200 mg Sulindac or an appropriate dose of other known anti-inflammatory drugs will be formulated in the IR form with 250 mg to 500 mg Metformin formulated in the IR form will be combined with ACE inhibitors such as 5 mg to 80 mg Quinapril or 2.5 mg to 20 mg Ramipril or 50 mg to 450 mg Captopril or 2.5 mg to 8 mg Trandolapril or 15 mg to 40 mg Benazepril or 25 mg to 80 mg Fosinopril or 5 mg to 80 mg Lisinopril or 15 mg to 60 mg Moexipril or 5 mg to 40 mg Enalapril formulated in the form of ER.

### Example 48

### FDC Formulations for Clinically Managing Insulin Resistance Syndrome or Metabolic Syndrome: Three Drug Combinations (IR/IR/ER): Anti-Inflammatory/Metformin/ARBs

200 mg Ibuprofen or 250 mg Naproxen-S odium or 325 mg of Aspirin or Aspirin-like drugs (e.g. Ascriptin, Ecotrin) or 50 mg to 400 mg Celecoxib or 200 mg Sulindac or an appropriate dose of other known anti-inflammatory drugs will be formulated in the IR form with 250 mg to 500 mg Metformin formulated in the IR form will be combined with (ARBs) such as 10 mg to 32 mg Candesartan or 150 mg to 300 mg Irbesartan or 25 mg to 40 mg Olmesartan or 25 mg to Losartan or 100 mg to 320 mg Valsartan or 25 mg to 80 mg Telmisartan or 250 mg to 800 mg Eprosartan formulated in the form of ER.

### Example 49

### FDC Formulations for Clinically Managing Insulin Resistance Syndrome or Metabolic Syndrome: Four Drug Combinations (IR/IR/ER/DR): Anti-Inflammatory/DPP-IV Inhibitors/ARBs/ACE Inhibitors

200 mg Ibuprofen or 250 mg Naproxen-S odium or 325 mg of Aspirin or Aspirin-like drugs (e.g. Ascriptin, Ecotrin) or 50 mg to 400 mg Celecoxib or 200 mg Sulindac or an appropriate dose of other known anti-inflammatory drugs will be formulated in the IR form with anti-type II diabetic DPP-IV inhibitors such as 12.5 mg to 100 mg Sitagliptin, 3 mg to 25 mg Alogliptin or 2.5 mg Linagliptin in an IR form with anti-hypertensive ARBs such as 2 mg to 32 mg Candesartan or 75 mg to 300 mg Irbesartan or 10 mg to 40 mg Olmesartan or 12.5 mg to Losartan or 40 mg to 320 mg Valsartan or 10 mg to 80 mg Telmisartan or 200 mg to 800 mg Eprosartan formulated in the ER form will be combined with lipid lowering Statins such as 80 mg Atorvastatin or 80 mg Provastatin or 40 mg Rosuvastatin or 40 mg Simvastatin formulated in the form of DR.

### Additional FDC Formulations for Clinically Managing Type 2 Diabetes and Prediabetes

### Example 50

50 mg to 400 mg Celecoxib in an IR form will be combined with 500 mg to 1000 mg Metformin in an IR or ER or DR form administered once or twice daily.

### Example 51

50 mg to 400 mg Celecoxib in an IR form will be combined with 500 mg to 1000 mg Metformin in an IR or ER or DR form and 80 mg to 160 mg Valsartan in a DR form administered once or twice daily.

### Example 52

50 mg to 400 mg Celecoxib in an IR form will be combined with 500 mg to 1000 mg Metformin in an IR or ER or DR form, 80 mg to 160 mg Valsartan in a DR form and 80 mg of Atorvastatin in an ER form administered once or twice daily.

### Example 54

50 mg to 400 mg Celecoxib in an IR form will be combined with 5 mg Linagliptin in IR form, 80 to 160 mg Valsartan in a DR form and 80 mg Atorvastatin in an ER form administered once or twice daily.

### Example 55

### Preparation of FDC Formulation of IR (Celecoxib) and ER (Metformin)

Preparation of multilayer tablets of 50 mg to 400 mg Celecoxib in an IR form and 250 mg to 2000 mg of Metformin HCl in an ER form.

The IR drug containing layer would contain, e.g., Celecoxib (50 mg to 400 mg), microcrystalline cellulose (50.0 mg), lactose monohydrate (78.0 mg), hydroxyl propyl cellulose (30.0 mg), Croscarmellose sodium (10.0 mg) and magnesium stearate (2.0 mg).

The ER drug containing layer would contain Metformin HCl (250 mg to 2000 mg), carnuba wax (290.0 mg), stearic acid (190.0 mg), silicon dioxide (10.0 mg), and magnesium stearate (2.0 mg).

The inert layer would contain carnauba wax (100.0 mg), dibasic calcium phosphate (58.0 mg), stearic acid (40 mg), and magnesium stearate (2.0 mg).

The subcoating would contain hydroxypropyl methyl cellulose (45.0 mg/tablet), polyethyl glycol 400 (12.0 mg/tablet) and purified water which will be removed during processing.

The ER coating contained Surelease (98.0 mg/tablet), hydroxyl methyl cellulose (98.0 mg/tablet) and purified water which would be removed during processing.

The IR drug containing the layer of the core tablet will be prepared as follows: Celecoxib will be blended with all the ingredients except Croscarmellose sodium and magnesium stearate and granulated with purified with water. The granulate will be dried and milled through a suitable screen. Croscarmellose sodium AND magnesium stearate will be added to the milled granules. The mixture will then be blended for 2 minutes.

The ER drug containing layer of the tablet will be prepared as follows: Metformin HCl and Carnauba wax will be mixed and granulated with a solution of stearic acid in ethyl alcohol.

The granulate will then be dried, milled through a suitable screen. Silicon dioxide and magnesium stearate will be screened and then added to the milled granules. The mixture will then be blended for another 2 minutes.

The inert layer will be prepared as follows: Carnauba wax and dicalcium phosphate will be mixed and granulated with a solution of stearic acid in ethyl alcohol. The granulate will then be dried, milled through a suitable screen. Magnesium stearate will be screened and then added to the milled granules. The mixture will then be blended for another 2 minutes.

The mixture will then be compressed into a multi-layer core tablet in the following sequence: ER containing layer, inert layer and IR drug containing layer using a multi-layer tablet press. Core tablets will then be subcoated.

The subcoating will be prepared by dissolving hydroxypropyl methyl cellulose and polyethylene glycol 400 in purified water and sprayed as a coating solution onto the multi-layer core tablet in a coating pan.

The ER coating will be prepared as follows: In a container purified water will be mixed with hydroxyl propyl methyl cellulose using a mixer until hydroxyl methyl cellulose was completely dissolved. The hydroxymethyl cellulose solution was then added to the surelease dispersion and mixed for 15 minutes. The resulting dispersion will be mixed during the entire coating process. Using the coating pan, the surelease/hydroxypropyl methyl cellulose dispersion will be sprayed onto the subcoated tablets until the required weight gain was achieved.

### CONCLUSION

Various modifications and variations of the described method and system of the invention will be apparent to those skilled in the art without departing from the scope and spirit of the invention. Although the invention has been described in connection with specific desired embodiments, it should be understood that the invention as claimed should not be unduly limited to such specific embodiments. Indeed, various modifications of the described modes for carrying out the invention that are obvious to those skilled in the fields of medicine, immunology, pharmacology, endocrinology, pharmaceutics or related fields are intended to be within the scope of the invention as defined by the following claims.

What is claimed is:

1. A pharmaceutical formulation for the treatment of metabolic syndrome comprising
(i) a therapeutically effective amount(s) of an active agent selected from the group consisting of at least one anti-diabetic drug; or
(ii) a therapeutically effective amount of at least one anti-hypertensive drug; or
(iii) a therapeutically effective amount of at least one anti-obesity drug; or
(iv) a therapeutically effective amount of at least one drug for the treatment of hyperlipidemia; or
(v) a combination of any of (i), (ii), (iii), and (iv); and
(vi) a therapeutically effective amount of at least one orally active anti-inflammatory drug.

2. The pharmaceutical formulation of claim 1, comprising a therapeutically effective amount(s) of a drug selected from the group consisting of at least one anti-diabetic drug, an anti-hypertensive drug, and a therapeutically effective amount of at least one orally active anti-inflammatory drug.

3. The pharmaceutical formulation of claim 1, comprising therapeutically effective amounts of at least two drugs selected from the group consisting of at least one an anti-hypertensive drug, at least one anti-diabetic drug, at least one drug for the treatment of hyperlipidemia, at least one anti-obesity drug, and at least one anti-inflammatory drug.

4. The pharmaceutical formulation of claim 1, comprising therapeutically effective amounts of at least one an anti-hypertensive drug, at least one anti-diabetic drug, at least one drug for the treatment of hyperlipidemia, at least one anti-obesity drug, and at least one anti-inflammatory drug.

5. The pharmaceutical formulation of claim 1, wherein the metabolic syndrome is Type II diabetes, comprising therapeutically effective amounts of a combination of one or more anti-inflammatory drug(s) and at least one drug used in the treatment of Type II Diabetes.

6. The pharmaceutical formulation of claim 1 wherein the metabolic syndrome is Type II diabetes, comprising a therapeutically effective amount(s) of a drug selected from the group consisting of at least one anti-diabetic drug and a blocker of Renin-Angiotensin System (RAS).

7. The pharmaceutical formulation of claim 1 for the treatment of hyperlipidemia, comprising therapeutically effective amounts of a combination of one or more anti-inflammatory drug(s) and one or more drugs selected from the group consisting of a statin, niacin, a fibrate, and combinations of any of the foregoing.

8. The pharmaceutical formulation of claim 1 for the treatment of obesity comprising therapeutically effective amounts of a combination of one or more anti-inflammatory drug(s) and one or more drugs selected from the group consisting of an anti-obesity drug, an anti-diabetic drug, and combinations of any of the foregoing.

9. The pharmaceutical formulation of claim 1 for the treatment of hypertension, comprising therapeutically effective amounts of one or more drugs selected from the group consisting of a beta blocker, a diuretic, an ACE inhibitor, an Angiotension II Receptor Blocker (ARBs), Calcium channel blocker (CCBs), a central agonist, a peripheral-acting adrenergic blocker, a direct vasodilator, a direct renin inhibitor, and combinations of any of the foregoing; and at least one anti-inflammatory drug.

10. The pharmaceutical formulation of claim 1, which is an oral solid dosage form.

11. The pharmaceutical formulation of claim 1, which is a dosage form selected from the group consisting of an oral dosage form, a parenteral dosage form, a transdermal dosage form, and a nasal dosage form.

12. The formulation of claim 1, wherein at least one of the drugs is in controlled or delayed release form.

13. A method of treating metabolic syndrome, comprising administering to a human patient having a condition selected from the group consisting of Type II diabetes, Hypertension, Hyperlipidemia and Obesity, a pharmaceutical formulation for the treatment of metabolic syndrome comprising
(i) a therapeutically effective amount(s) of a drug selected from the group consisting of at least one anti-diabetic drug; or
(ii) a therapeutically effective amount of at least one anti-hypertensive drug; or
(iii) a therapeutically effective amount of at least one anti-obesity drug; or
(iv) a therapeutically effective amount of at least one drug for the treatment of hyperlipidemia; or
(v) a combination of any of (i), (ii), (iii), and (iv); and
(vi) a therapeutically effective amount of at least one orally active anti-inflammatory drug.

14. The method of claim 13, wherein the patient is diabetic or prediabetic, comprising administering a therapeutically effective amount(s) of a drug selected from the group consisting of at least one anti-diabetic drug, a blocker of Renin-Angiotensin System (RAS), and a combination of the foregoing, together with a therapeutically effective amount of at least one orally active anti-inflammatory drug.

15. The method of claim 13, wherein one or more of an ACE inhibitor, an angiotension II receptor blocker (ARBs), a DPPIV inhibitor, at least one anti-diabetic drug, a blocker of Renin-Angiotensin System (RAS), together with a therapeutically effective amount of at least one orally active anti-inflammatory drug, are administered to the patient.

16. The method claim 13, wherein the drugs are administered to the patient on a chronic basis.

17. The method of claim 14, wherein the drugs are administered as personalized medicines to clinically manage Type II diabetes or prediabetes in stratified groups of human patients based on their cardiometabolic risk factor profiles.

18. The method of claim 14, wherein the drugs are administered as personalized medicines to clinically manage Type II diabetes or prediabetes groups of patients stratified for lack of adequate therapeutic efficacy with prior treatments.

19. The method of claim 14, further comprising concomitantly administering insulin to the patient to achieve better blood glucose control.

20. The method of claim 14, wherein dosage is adjusted on the basis of glucose measurements.
